(19) United States
(12) Patent Application Publication
(10) Pub. No.: US 2010/0233226 A1
(43) Pub. Date: Sep. 16, 2010

(54) DRUG-ELUTING NANOWIRE ARRAY

(75) Inventors: Etienne Ferain, Masnuy-Saint-Jean (BE); Delphine Magnin, Luttre (BE); Sophie Demoustier-Champagne, Bossiere (BE); Marie-Anne Thil, Bruxelles (BE); Jean Delbeke, Mouscron (BE); Ides Colin, Beauvechain (BE)

Correspondence Address: KNOBBE MARTENS OLSON & BEAR LLP, 2040 MAIN STREET, FOURTEENTH FLOOR, IRVINE, CA, 92614, US

(73) Assignee: Université Catholique de Louvain, Louvain-la-Neuve (BE)

(21) Appl. No.: 12/681,755

(22) PCT Filed: Oct. 14, 2008
(86) PCT No.: PCT/EP2008/063803

§ 371 Date: Apr. 5, 2010

(30) Foreign Application Priority Data
Oct. 15, 2007 (EP) 07118428.7

(51) Int. Cl. A61F 2/00; A61N 1/04; C25D 5/02; A61K 31/573; A61K 39/395; A61K 31/616; A61K 31/7088; A61K 38/18; A61K 38/17; A61K 38/43; A61P 35/00; A61P 29/00; A61P 3/02; A61P 39/06; A61P 25/16; A61P 25/18; A61P 25/06; A61P 25/08

(52) U.S. Cl. 424/422; 607/137; 607/116; 607/118; 205/122; 216/20; 514/180; 424/142.1; 424/133.1; 424/134.1; 514/165; 514/44 A; 514/12; 514/8; 424/94.1; 977/762

(57) ABSTRACT

The present invention relates to a nanowire array (15, 16) for electrically-controlled elution of a therapeutic composition (5) comprising a plurality of nanoscopic-sized wires (12, 12), nanowires, attached to an electrically conducting solid support (7), said nanowires formed from electroactive conjugated polymer (4) containing or doped with said therapeutic composition (5) coated over a plurality of nanoscopic sized electrically conducting protrusions (8). It also relates to a method for preparing a nanowire array and an electrode.

### FIELD OF THE INVENTION

The present invention relates a nanowire array and an electrode comprising the same for local release of a therapeutic composition avoiding and controlling early morphological changes (for example fibrosis) in and around the nerve and the electrode to improve its implantation. This invention allows the release drugs or chemicals with a high degree of precision in the localization, quantity and time of delivery.

### BACKGROUND OF THE INVENTION

Neurological conditions such as spinal cord injuries result in dramatic harmful paralysis for several thousands people every year. Attempts to improve the patient quality of life by functional electrical stimulation (FES) have been carried out over the last 30 years with increasing success. This led to the development of new implanted stimulators and to the engineering of innovative peripheral prosthetic devices in order to optimise the quality of neural stimulation, but also of recorded neural signals toward various paradigms of closed loop systems. Besides the rehabilitation of para- and tetraplegic patients, FES has also been used to restore functions in incontinent and sensory impaired patients. Hence, the application field of FES is particularly broad.

One of the main challenges that must be faced in this field of research is to optimise the interface nerve-prosthetic device in order to reduce disturbing interference to a minimum level. Careful attention has been paid to the upgrading of peripheral electrodes. Their performance has been improved by increasing the number and the geometry of metallic dots and networks in contact with the nerve. This can be achieved by application of original methods of metal deposition, as previously described. Most issues related to tissue biocompatibility have been temporally solved through the selection of specific materials that were shown to be biologically relatively inert.

Another major breakthrough in the field results from the use of spiral cuff electrodes. Due to their self-sizing properties, spiral cuff electrodes are expected to accommodate nerve swelling and consequently to limit mechanical lesions and vascular injuries. Thus, because of their physical properties, spiral cuff electrodes were proven to be suitable for long-term implantation. The clinical applications of cuff electrodes are numerous and include sacral nerve root stimulation to restore bladder function, peripheral nerve stimulation in para and tetraplegic patients, as aforementioned, stimulation of the phrenic nerve for diaphragm pacing to provide respiratory support, stimulation of the vagus nerve in epileptic and some depressive patients, and stimulation of the optic nerve to improve visual perception in blind patients. Nevertheless, one must admit that the technology applied to neural electrodes still remains suboptimal. Limitation of efficient neural electrode use is related to their propensity to induce morphological changes within the nerve, as soon as they are implanted. They include nerve reshaping, growth of surrounding connective tissue, fibrosis of the epineurium (the external compartment of the nerve), and loss of myelinated fibres followed by regeneration. These morphological changes are likely to cause alterations in functional electrode performances. Thus, electrophysiological instability is a complication that arises immediately after spiral cuff implantation as a consequence of morphological alterations.

Evidence accumulated over the recent years indicates that variable shifts in thresholds, unstable recordings, and decreased reproducibility in strength of stimulated motor responses may arise from alterations in the structural integrity of implanted nerves. Nerve reshaping is actually observed as early as 18 hours after implantation, whereas electrode encapsulation and fibrosis start from day seven and evolve onwards. The commonly described fibrotic reaction is preceded by an important epineurial inflammatory and oedematous reaction. Indeed, the mechanical stress associated with the surgical procedure is known to induce microvascular lesions and increases vascular permeability. The resulting epineurial swelling due to inflammation and interstitial oedema may affect the tissue to electrode contact and in turn the electrode efficacy. This early reaction is followed by the progressive deposit of connective tissue layer that become denser and tend to merge with the perineurium (the connective tissue that directly protects neural fascicles). This process tends to make the perineurium thicker and stronger and may contribute to protect the endoneurium from external aggressions in order to safeguard endoneurial functional properties. Morphological changes that occur at long-term after electrode cuff implantation could therefore be viewed as beneficial; at least to safeguard neural functions that directly depend on the integrity of the endoneurial compartment. Therapeutic interaction with the nerve function, however and in the shorter term, unstable electrophysiological properties are largely unsatisfactory and a maximal functional efficiency should be reached as soon as the electrode is fixed. Limiting the inflammation but preserving the external fibrotic reaction could be a reasonable goal since electrophysiological instability is expected to be reduced, while maintaining a better electrode anchorage and reducing rubbing forces. The acute inflammatory reaction and the expansion of connective tissue in the epineurium are regulated by a set of cytokines and factors that interact with each other in a complex network. For instance, TNF-alpha is a pro-inflammatory cytokine minimally expressed in the intact peripheral nervous system, but up-regulated within the endoneurium after injury. It represents one of the best targets when aiming at improving the nerve/cuff electrode interface. TNF-alpha expression has been shown to increase immediately after cuff-implantation and remains elevated, mostly within the epineurium, up to one month after surgery. Increased expression of TNF-alpha is associated with demyelination, degeneration, inflammation, and ectopic electrophysiological activities in the sciatic nerve. Modulating some aspects of the nerve reaction related for example to the expression of locally-produced cytokines could therefore be the key for a significant improvement of the quality of nerve recordings and FES. In accordance with this, a systemic treatment with anti-TNF-alpha antibodies has been shown to reduce the early inflammatory reaction following cuff implantation.

Prior art discloses electrodes for drug delivery. For example, U.S. Pat. No. 5,422,246 (Koopal et al.) describes an electrode coated with a polypyrrole film having a redox enzyme bound thereto. Polypyrrole coating is prepared by chemical polymerisation within a nanoporous polymeric membrane. The use of conjugated polymer for drug release is know in the art, see for example Cui X et al. (Journal of Controlled Release (2006), 110(3) 531-541) which described a film of polypyrrole for electrochemically controlled release of bioactive molecules. Cui X et al (Biomaterials, 2003, 24(5), pages 777-787) describes a peptide-loaded polypyrrole coating that can be made to attract neurons selectively and reduce the electrode interface impedance by providing charge exchangers, which features are short lived. Cui X et al (Journal of Biomaterials Research, 2001, 56(2), pages 261-272) discloses that a rough surface disposed with a polypyrrole/biomolecule coating, that promotes selective adhesion of different cell types. He W et al (Biomaterials, 2005, 26(16), pages 2983-2990) describes the use of a polypyrrole coating in order to improve the biocompatibility of silicon oxide. Wadhwa et al (Journal of controlled release, 2006, 110(3), pages 531-541) describes the release of dexamethasone to reduce the inflammatory reaction around the electrode. Konitturi Kyosti et al (J. Electroanal Chem, 1998, 453(1-2), pages 231-238) describes a polypyrrole/sodium tosylate film disposed on an electrode. US 2006/214156 describes the use of nanotubes (typically carbon) and nanowires embedded in hybrid material to build small plastic transistors.

The invention differs from the prior art either by the configuration of the electrode or the use of polymeric substance embedded with a therapeutic composition coated over nanoscopic metallic protrusions. The aim of the invention is to provide nanowire array and an electrode comprising the same able to locally release drugs avoiding early morphological changes near an implanted electrode.

### SUMMARY OF THE INVENTION

It is an object of the invention to provide a nanowire array able to release a therapeutic composition, which array comprises a plurality of nanowires formed from electroactive conjugated polymer which is doped with a therapeutic composition.

The term “nanowire array” as used in the present invention relates to a structure formed from a plurality of wires each wire having a nanoscopic size. According to the present invention, a wire is an elongate structure having nanoscale (nm to μm) dimensions. It may have aspect ratio comprised between 0.4 and 2000. The term “aspect ratio” relates to the ratio between the length and the width of the wire. It is made at least partly from an electroactive conjugated polymer and preferably has an essentially cylindrical shape. Their width is comprised between 10 nm and 10 μm.

The term “electroactive conjugated polymer” as used in the present invention refers to conjugated polymers having the ability to undergo reversible redox reaction when a voltage is applied to them. Conjugated polymers as used in the invention can be polymers or copolymers based on heterocycle moiety as monomers, aniline and substituted aniline derivatives, cyclopentadiene and substituted cyclopentadiene derivatives, phenylene or substituted phenylene derivatives, pentafulvene and substituted pentafulvene derivatives, acetylene and substituted acetylene derivatives, indole and substituted indole derivatives, carbazole and substituted carbazole derivatives or compounds based on formula (I) or (II) wherein n is an integer greater than 1, 2, 3, 4, or 5, or is between 1 and 1000, 5 000, 10 000, 100 000, 200 000, 500 000 or 1 000 000 or higher, X is selected from the group consisting of —NR¹—, O, S, PR², SiR⁵R⁶, Se, AsR³, BR⁴ wherein R and R′ which can be identical or notare independently selected from the group consisting of, linked or not, are alkyl, aryl, hydroxyl, alkoxy or R and R′ together with the carbon atoms to which they are attached form a ring selected from aryl, heteroaryl, cycloalkyl, heterocyclyl, wherein R¹, R², R³ and R⁴, R⁵ and R⁶ are independently selected from the group consisting of hydrogen, alkyl or aryl group and wherein A and A′ can are independently selected from the group consisting of be heterocycle, heterocyclyl, alkenyl, alkynyl or aromatic ring and wherein A and A′ can be identical or not.

In a preferred embodiment, the conjugated polymers are based on heterocycle moiety as monomers such as pyrrole and substituted pyrrole derivatives, furan and substituted furan derivatives, thiophene and substituted thiophene derivatives, phosphole and substituted phosphole derivatives, silole and substituted silole derivatives, arsole and substituted arsole derivatives, borole and substituted borole derivatives, selenole and substituted selenole derivatives or aniline and substituted aniline derivatives.

In a preferred embodiment, the conjugated polymers are based on pyrrole and substituted pyrrole derivatives.

According to the present invention, the electroactive conjugated polymer is doped with a therapeutic composition or drug that is locally released upon further electrical stimulation. The therapeutic composition may comprise bioactive molecules of interest including, for example, nutritional substances such as vitamins; active compounds such as anticancer drugs, antipsychotic, antiparkinsonian agents, antiepileptic agents, antimigraine agents; nucleic acids such as nucleotides, oligonucleotides, antisense oligonucleotides, DNA, RNA and mRNA; amino acids and natural, synthetic and recombinant proteins, glycoproteins, polypeptides, peptides, enzymes; antibodies, hormones, cytokines and growth factors. Preferably, the therapeutic composition comprises one or more anti-inflammatory agents. More preferably, the therapeutic composition comprises one or more anti-TNF-alpha agents such as adalimumab, infliximab, etanercept, certolizumab pegol, and golimumab; one or more steroidal anti-inflammatory agents such as dexamethasone disodium; one or more non-steroidal anti-inflammatory agents like aceclofenac, acemetacin, aspirin, celecoxib, dexibuprofen, dexketoprofen, diclofenac, diflunisal, etodolac, etoricoxib, fenbrufen, fenoprofen, flurbiprofen, ibuprofen, indomethacin, ketoprofen, ketorolac trometamol, lumiracoxib, mefanamic acid, meloxicam, nabumetone, naproxen, nimesulide, oxaprozin, parecoxib, phenylbutazone, piroxicam, proglumetacin, sulindac, tenoxicam, and tiaprofenic acid.

Another embodiment of the invention is an electrode provided with a nanowire array in electrical contact with the electrode. Said electrode is able to release the therapeutic composition upon stimulation. The electrode typically comprises metallic contacts wherein a nanowire array according to the invention is disposed onto at least part of the metallic contacts. In a preferred embodiment, the electrode is an implantable self-sizing spiral cuff electrode.

Different releasing surfaces can be placed on the same electrode, each releasing a different drug or therapeutic composition as required by the specific application.

The term “self-sizing spiral cuff electrode” as used in the invention refers to an electrode wherein the spiral cuff naturally wraps around the nerve to form a tube. Due to its self-sizing properties, the spiral cuff electrode is expected to accommodate nerve swelling and thus to avoid mechanical lesion, as well as vascular sequels to the nerve.

Preferably, the metallic contacts are made from noble metals such as platinum or gold. Contacts within the cuff may be cut from platinum foils and welded to stainless steel leads or alternatively contacts and/or leads can be formed by metal deposition on appropriately shaped silicone rubber. These contacts may then be inserted between two sheets of silicone rubber, one being stretched, before being bonded with the silicone elastomer to create a self-curling spiral cylinder (Naples et al. IEEE Trans. Biomed. Eng. 35, 905-916).

Another aspect of the invention is method for the preparation of a nanowire array that elutes a therapeutic composition comprising the steps of:

(a) depositing a layer of polymeric matrix onto at least part of an electrically conducting solid support,
(b) creating pores in the layer of polymeric matrix by track-etching so forming a polymeric nanoporous layer,
either:
(c) electrodepositing an electrically conducting material within the pores of the polymeric nanoporous layer,
(d) dissolving the polymeric nanoporous layer to form electrically conducting protrusions, and
(e) electropolymerising an electroactive conjugated polymer 4 doped with therapeutic composition;
   or:
(C) electropolymerising an electroactive conjugated polymer within the pores of the polymeric nanoporous layer, so creating hollow nanoscopic sized wires,
(D) applying the therapeutic composition to the hollow of the wires, and
(E) electropolymerising a layer of electroactive conjugated across the open end of the nanoscopic sized wires, to form a cap;
   so forming a nanowire array.

The inventors have found that the presence of nanowires strongly influences the electroactivity of the film. Particularly, the deposition of electroactive conjugated polymer on the nanostructured metal surface i.e. formed from nanoscopic sized electrically conducting protrusions, increases activity of the conjugated polymer, which phenomenon is linked to an increase in electrical conductivity of the polypyrrole. Moreover, the nanostructuring improves adherence of the polymer and increases the specific surface of the electrode. Thus, it is possible not only to increase dramatically the quantity of therapeutic compound that could be released by the polymer, but the local current density of the electrode surface can be adapted to specific needs, simply by tuning the density of nanowires or holes on the electrode. Due to the large surface area of an electrode incorporating the nanostructured wires so formed, the redox response is stronger compared to conventional macroelectrodes. The inventors have further found that release by the array of therapeutic composition follows a kinetic order of one; this has advantages of an easy calibration of the system, by establishing a relation between the potential or current and the amount of therapeutic molecules released. Therefore, at any time, the amount of remaining therapeutic molecules on the nanowires array can be determined.

The local density of nanowires on the electrodes is adaptable by, for instance, changing the density of pores of the polymeric nanoporous layer. Adapting the local density of nanowires allows the local current density to be adapted on the conducting solid support 7. Compared with non-wire array electrodes, tuning the local current density allows compensation for the ‘edge effect’ (high currents on the edges of the electrodes) observed on flat electrodes.

Moreover, creating nanostructures that are bound to an electrically conducting solid support that has a millimeter or micrometer dimensions maintains the benefits of nanostructuring without implanting nano-sized objects that can freely migrate within a body.

The electrical command for release control can be carried out via the pre-existing circuits on the implantable medical device and a wide variety of electrodes can be developed since several drugs can be added as hydrated ions during the electropolymerisation step.

The electrodes according to the invention can be used in several medical applications, including, but not limited to vagus nerve stimulation, deep brain stimulation, and prosthetic devices, on brain interfaces, oncology or inflammatory diseases.

### BRIEF DESCRIPTION OF THE DRAWINGS

FIG. 1A: Three dimensional representation of a nanowire array of the present invention.

FIG. 1B: Transverse cross section across plane X-X′ of a nanowire array of the present invention, whereby nanowires of the array are formed from electrically conducting protrusions coated with electroactive conjugated polymer doped with therapeutic composition.

FIG. 2A: Three dimensional representation of a nanowire array of the present invention.

FIG. 2B: Transverse cross section across plane X-X′ of a nanowire array of the present invention, whereby nanowires of the array are formed from electroactive conjugated polymer fashioned into containers holding therapeutic composition.

FIG. 3: Redox process at the basis of drug release from polypyrrole.

FIG. 4A to 4D: Steps for the preparation of a nanowire array of the invention, showing four stages for preparing nanowires formed from electrically conducting protrusions coated with electroactive conjugated polymer doped with therapeutic composition indicated on a transverse cross-section.

FIG. 5A to 5D: Steps for the preparation of a nanowire array of the invention, showing four stages for preparing nanowires formed from electroactive conjugated polymer fashioned into containers holding therapeutic composition indicated on a transverse cross-section.

FIG. 6: Schematic representation of the apparatus and method employed to form a self-curling cuff incorporating electrodes disposed with a nanowire array of the invention.

FIG. 7. A plan view of side view of unstretched sheet bearing four contact electrodes and wires.

FIG. 8 Schematic representation of the steps of forming a cuff electrode.

FIG. 9. Scanning electron microgram of an array of nano-sized platinum protrusions.

FIG. 10 Scanning electron microgram of a nanowire array, whereby the coating comprises a mixture of polypyrrole and dexamethasone.

FIG. 11 Graphic illustrating the kinetics of active release of dexamethasone based on the number of cycle of electrical stimulation and passive release kinetics as a function of time where 1 cycle corresponds to 1 minute.

FIG. 12 Graphic illustrating the influence of film thickness of polypyrrole on the release of dexamethasone.

### DETAILED DESCRIPTION OF THE PREFERRED EMBODIMENTS

Unless defined otherwise, all technical and scientific terms used herein have the same meaning as is commonly understood by one skilled in the art. All publications referenced herein are incorporated by reference thereto. All United States patents and patent applications referenced herein are incorporated by reference herein in their entirety including the drawings.

The articles “a” and “an” are used herein to refer to one or to more than one, i.e. to at least one of the grammatical object of the article. By way of example, “a nanowire” means one nanowire or more than one nanowire.

Throughout this application, the term “about” is used to indicate that a value includes the standard deviation of error for the device or method being employed to determine the value.

The recitation of numerical ranges by endpoints includes all integer numbers and, where appropriate, fractions subsumed within that range (e.g. 1 to 5 can include 1, 2, 3, 4 when referring to, for example, a number of electrodes, and can also include 1.5, 2, 2.75 and 3.80, when referring to, for example, measurements). The recitation of end points also includes the end point values themselves (e.g. from 1.0 to 5.0 includes both 1.0 and 5.0)

The present invention relates to a drug-eluting nanowire array that releases an active compound when a current is applied thereto. The nanowire array has particular use in the field of locally drug delivery. When provided as part of an electrode, the array can control early morphological changes in a nerve and around the electrode with the aim of achieving an improved functional efficiency, especially early after electrode implantation. The present invention also relates to an electrode on which the nanowire array is disposed. It also relates to a method of preparation of the array and of the electrode.

According to one embodiment, the present invention provides a nanowire array able to locally release a therapeutic composition. The nanowire array comprises a plurality of nanoscopic-sized wires (nanowires) formed from electroactive conjugated polymer containing or doped with said therapeutic composition.

The nanoscopic sized wire present in an array is available in two main configurations. In a preferred first embodiment, the nanoscopic sized wire present in an array (1) as a conductive (e.g. metal) nanosized wire coated with electroactive conjugated polymer doped with therapeutic composition. In a second embodiment, the nanoscopic sized wire present in an array (1) as a hollow nanoscopic sized wire formed from electroactive conjugated polymer, containing therapeutic composition.

Reference is made in the description below to the drawings that exemplify particular embodiments of the invention; they are not at all intended to be limiting. The skilled person may adapt the device and method and substitute components and features according to the common practices of the person skilled in the art.

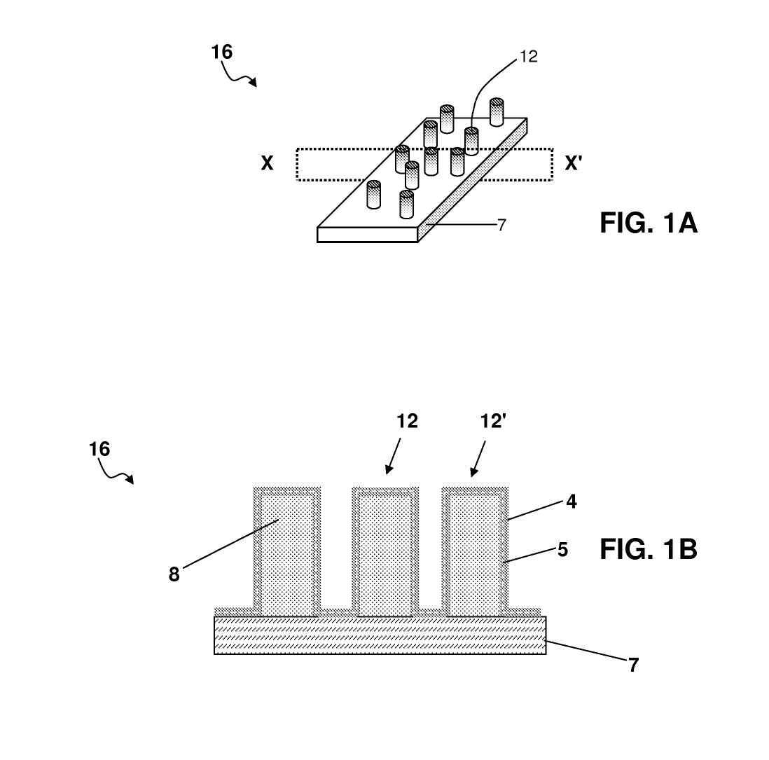

A first configuration of the nanowire array 16 is shown in FIGS. 1A and 1B and comprises a plurality of nanosized protrusions 8 that are conductive (e.g. metallic) wires attached to a solid, electrically conducting support 7, coated with electroactive conjugated polymer 4 which has been doped with therapeutic composition 5, so forming the nanoscopic sized wires 12, 12′ of the invention.

The protrusions 8 may be made from any suitable conducting material such as copper, titanium, gold, silver, platinum, palladium, bismuth, or nickel. It is preferably made from noble metal such as platinum or gold.

A protrusion 8 of the invention has an elongate shape generally, but not always, having a length longer than the width. Preferably it has a cylindrical or essentially cylindrical shape, in which case the width has the same meaning as diameter. According to one aspect of the invention, a protrusion 8 may have a width of 10 nm, 50 nm, 100 nm, 200 nm, 300 nm, 400 nm, 500 nm, 600 nm, 700 nm, 800 nm, 900 nm, 1000 nm (1 micron), 2 microns, 3 microns, 4 microns, 5 microns, 6 microns, 7 microns, 8 microns, 9 microns, 10 microns or a value in the range between any two of the aforementioned values, less the total thickness of the electroactive conjugated polymer 4 coating. Preferably a nanoscopic sized wire has a width between 10 nm and 10 microns, preferably between 10 nanometers and 1 micron, more preferably between 10 and 500 nanometers, less the total width of the electroactive conjugated polymer 4 coating.

In another embodiment, a protrusion 8 may have an aspect ratio (length/width ratio) of 0.4, 1, 5, 10, 50, 100, 200, 300, 400, 500, 600, 700, 800, 900, 1000, 1200, 1400, 1600, 1800 2000 or a value in the range between any two of the aforementioned values. Preferably a protrusion 8 has an aspect ratio between 0.4 and 2000, preferably between 10 and 2000 and more preferably between 100 and 2000.

Preferably, the protrusions 8 adopt suitable size and shape to provide the nanoscopic sized wires after coating, which wires have dimensions as defined later below.

A protrusion 8 of the invention is coated with electroactive conjugated polymer 4 by electropolymerisation. The thickness of the coating can be controlled readily by the coating process (described below), the desired thickness being determined by the size of the protrusion 8, and the quantity and rate of delivery of the therapeutic composition 5 required. According to one aspect of the invention, the thickness of a coating of electroactive conjugated polymer 4 may be 1 nm, 5 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 micron or a value in the range between any two of the aforementioned values. Preferably the thickness of the coating is between 1 nm and 1 micron, preferably between 1 and 100 nanometers, more preferably between 1 and 50 nanometers.

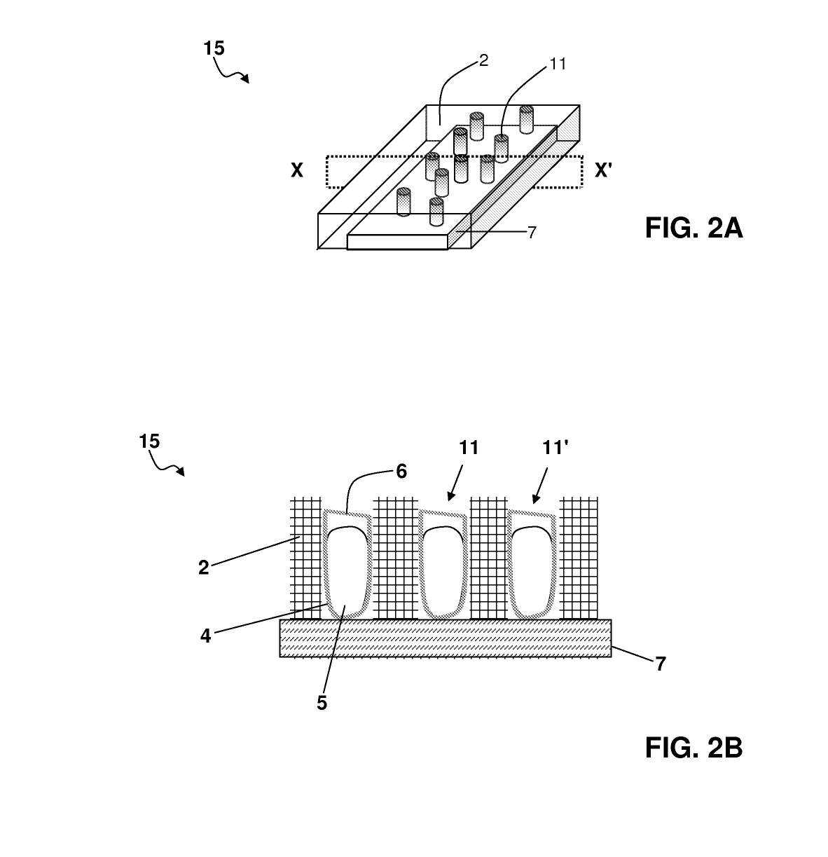

A second configuration of the nanowire array 15, depicted in FIGS. 2A and 2B, comprises a plurality of hollow nanoscopic sized wires 11, 11′ made from electroactive conjugated polymer 4 attached to an electrically conducting solid support 7. The hollow in each wire 11, 11′ contains therapeutic composition 5. The entrance to the hollow in the wire is capped 6 with a layer of electroactive conjugated polymer. The spaces between the wires 11, 11′ are disposed with a supporting matrix, which is a polymeric matrix 2.

The nanoscopic sized wires 11, 11′, 12, 12′ of the nanowire array 15, 16 are arranged on an electrically conducting solid support 7. The nanoscopic sized wires 11, 11′, 12, 12′ of the nanowire array 15, 16 are preferably mechanically attached to the electrically conducting solid support 7. The nanoscopic sized wires 11, 11′, 12, 12′ of the nanowire array 15, 16 are preferably in electrical contact with the electrically conducting solid support 7. The support 7 may be formed from any suitable electrically conducting material such as copper, titanium, gold, silver, platinum, palladium, bismuth, nickel, stainless steel; preferably it is made from noble metal such as platinum or gold. A nanoscopic sized wire 11, 11′, 12, 12′, being elongate and having longitudinal axis is preferably oriented essentially perpendicular to one surface of the support 7. The support 7, may be electrically connected to one or more electrically conducting wires for stimulatory release of the therapeutic composition 5.

The density (number of nanowires/cm²) of nanoscopic sized wires (nanowires) 11, 11′, 12, 12′ present in a nanowire array 15, 16 may be 5 nanowires/cm², 10 nanowires/cm², 10² nanowires/cm², 10³ nanowires/cm², 10⁴ nanowires/cm², 10⁵ nanowires/cm², 10⁶ nanowires/cm², 10⁷ nanowires/cm², 10⁸ nanowires/cm², 10⁹ nanowires/cm², and 10¹⁰ nanowires/cm² or a value in the range between any two of the aforementioned values. Preferably the nanowires density is between 10⁵ pores/cm² to 10⁹ pores/cm², preferably between 10⁸ and 10⁹ pores/cm².

According to one aspect of the invention, the density of nanoscopic sized wires is not uniform on the electrically conducting solid support 7. The ratio of the total area to the area of the electrically conducting solid support may greatest at the centre of the array, allowing compensation for non-uniform current density at the array surface. According to one aspect of the invention, the density of nanoscopic sized wires (nanowires) 11, 11′, 12, 12′ present in a nanowire array 15, 16 is greater in a subregion of the electrically conducting solid support 7. According to another aspect of the invention, a subregion of the electrically conducting solid support 7 has a density of nanoscopic sized wires (nanowires) 11, 11′, 12, 12′ that is at least 10%, 20%, 30%, 40%, 50%, 60%, or 70% higher than outside the subregion. In a preferred aspect of the invention, the subregion occupies no more than 10%, 20%, 30%, 40%, 50%, 60%, 70%, 80% or 90% of the coated surface of the electrically conducting solid support 7, or a value in the range between any two of the aforementioned values, preferably between 30 and 80%. In a preferred aspect of the invention, the subregion is located towards the centre of the electrically conducting solid support 7. Advantageously, subregion disposed with a higher density of nanoscopic sized wires reduces the electrode ‘edge effect’ (high currents on the edges of the electrodes) observed for flat electrodes.

A nanoscopic sized wire 11, 11′, 12, 12′ of the invention has an elongate shape generally, but not always having a length longer than the width. Preferably it has a cylindrical or essentially cylindrical shape, in which case the width has the same meaning as diameter. According to one aspect of the invention, a nanoscopic sized wire 11, 11′, 12, 12′ may have a width of 10 nm, 50 nm, 100 nm, 200 nm, 300 nm, 400 nm, 500 nm, 600 nm, 700 nm, 800 nm, 900 nm, 1000 nm (1 micron), 2 microns, 3 microns, 4 microns, 5 microns, 6 microns, 7 microns, 8 microns, 9 microns, 10 microns or a value in the range between any two of the aforementioned values. Preferably a nanoscopic sized wire has a width between 10 nm and 10 microns, preferably between 10 nanometers and 1 micron, more preferably between 10 and 500 nanometers.

In another embodiment, nanoscopic sized wire 11, 11′, 12, 12′ may have an aspect ratio (length/width ratio) of 0.4, 1, 5, 10, 50, 100, 200, 300, 400, 500, 600, 700, 800, 900, 1000, 1200, 1400, 1600, 1800 2000 or a value in the range between any two of the aforementioned values. Preferably a nanoscopic sized wire 11, 11′, 12, 12′ has an aspect ratio between 0.4 and 2000, preferably between 10 and 2000 and more preferably between 100 and 2000.

An electroactive conjugated polymer 4 refers to conjugated polymers having the ability to undergo reversible redox reaction when a voltage is applied to them. Conjugated polymers as used in the invention can be polymers or copolymers based on heterocycle moiety as monomers, aniline and substituted aniline derivatives, cyclopentadiene and substituted cyclopentadiene derivatives, phenylene or substituted phenylene derivatives, pentafulvene and substituted pentafulvene derivatives, acetylene and substituted acetylene derivatives, indole and substituted indole derivatives, carbazole and substituted carbazole derivatives or compounds based on formula (I) or (II) wherein n is an integer greater than or equal to 1, 2, 3, 4, or 5, or is between 1 and 1000, 5 000, 10 000, 100 000, 200 000, 500 000 or 1 000 000 or higher, X is selected from the group consisting of —NR¹—, O, S, PR², SiR⁵R⁶, Se, AsR³, BR⁴ wherein R and R′ are independently selected from the group consisting of, alkyl, aryl, hydroxyl, alkoxy or R and R′ together with the carbon atoms to which they are attached form a ring selected from aryl, heteroaryl, cycloalkyl, heterocyclyl, wherein R¹, R², R³, R⁴, R⁵ and R⁶ are independently selected from the group consisting of hydrogen, alkyl or aryl group and wherein A and A′ are independently selected from the group consisting of heterocyclyl, alkenyl, alkynyl or aromatic ring.

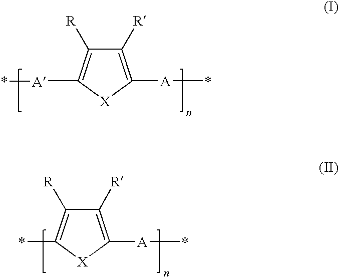

The term copolymers as used herein refers to polymers derived from at least two different monomeric species. Copolymers can be alternating, periodic, statistical, random or block copolymers.

The term “alkyl” by itself or as part of another substituent refers to a hydrocarbyl radical of Formula CₙH₂ₙ₊₁ wherein n is a number greater than or equal to 1. Generally, alkyl groups of this invention comprise from 1 to 6 carbon atoms, preferably from 1 to 4 carbon atoms, more preferably from 1 to 3 carbon atoms, still more preferably 1 to 2 carbon atoms. Alkyl groups may be linear or branched and may be substituted as indicated herein. When a subscript is used herein following a carbon atom, the subscript refers to the number of carbon atoms that the named group may contain. Thus, for example, C₁₋₄ alkyl means an alkyl of one to four carbon atoms. C₁₋₆alkyl includes all linear, or branched alkyl groups with between 1 and 6 carbon atoms, and thus includes methyl, ethyl, n-propyl, i-propyl, butyl and its isomers (e.g. n-butyl, i-butyl and t-butyl); pentyl and its isomers, hexyl and its isomers.

The term “aryl” as used herein refers to a polyunsaturated, aromatic hydrocarbyl group having a single ring (i.e. phenyl) or multiple aromatic rings fused together (e.g. naphtyl). or linked covalently, typically containing 5 to 12 atoms; preferably 6 to 10, wherein at least one ring is aromatic. The aromatic ring may optionally include one to two additional rings (either cycloalkyl, heterocyclyl or heteroaryl) fused thereto. Aryl is also intended to include the partially hydrogenated derivatives of the carbocyclic systems enumerated herein. Non-limiting examples of aryl comprise phenyl, biphenylyl, biphenylenyl, 5- or 6-tetralinyl, 1-, 2-, 3-, 4-, 5-, 6-, 7- or 8-azulenyl, naphthalen-1- or -2-yl, 4-, 5-, 6 or 7-indenyl, 1-2-, 3-, 4- or 5-acenaphtylenyl, 3-, 4- or 5-acenaphtenyl, 1-, 2-, 3-, 4- or 10-phenanthryl, 1- or 2-pentalenyl, 4- or 5-indanyl, 5-, 6-, 7- or 8-tetrahydronaphthyl, 1,2,3,4-tetrahydronaphthyl, 1,4-dihydronaphthyl, 1-, 2-, 3-, 4- or 5-pyrenyl.

The aryl ring can optionally be substituted by one or more substituent(s). An “optionally substituted aryl” refers to an aryl having optionally one or more substituent(s) (for example 1 to 5 substituent(s)), for example 1, 2, 3 or 4 substituent(s) at any available point of attachment selected independently in each incidence. Unless provided otherwise, non-limiting examples of such substituents are selected from halogen, hydroxyl, oxo, nitro, amino, cyano, alkyl, cycloalkyl, alkenyl, alkynyl, cycloalkylalkyl, C₁₋₄alkylamino, C₁₋₄ dialkylamino, alkoxy, aryl, heteroaryl, arylalkyl, haloalkyl, haloalkoxy, alkoxycarbonyl, alkylcarbamoyl, heteroarylalkyl, alkylsulfonamide, heterocyclyl, alkylcarbonylaminoalkyl, aryloxy, alkylcarbonyl, acyl, arylcarbonyl, carbamoyl, alkylsulfoxide, alkylcarbamoylamino, sulfamoyl, N—C₁₋₄-alkylsulfamoyl or N,N—C₁₋₄ dialkylsulfamoyl, —SO₂R^{c}, alkylthio, carboxyl, and the like, wherein R^{c} is C₁₋₄alkyl, haloalkyl, C₃₋₆cycloalkyl, C₁₋₄ alkylsulfonamido or optionally substituted phenylsulfonamido.

The term “heteroaryl” as used herein by itself or as part of another group refers but is not limited to 5 to 12 carbon-atom aromatic rings or ring systems containing 1 to 2 rings which are fused together or linked covalently, typically containing 5 to 6 atoms; at least one of which is aromatic in which one or more carbon atoms in one or more of these rings can be replaced by oxygen, nitrogen or sulfur atoms where the nitrogen and sulfur heteroatoms may optionally be oxidized and the nitrogen heteroatoms may optionally be quaternized. Such rings may be fused to an aryl, cycloalkyl, heteroaryl or heterocyclyl ring. Non-limiting examples of such heteroaryl, include: pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl.

The term “cycloalkyl” as used herein is a cyclic alkyl group, that is to say, a monovalent, saturated, or unsaturated hydrocarbyl group having 1 or 2 cyclic structure. Cycloalkyl includes all saturated hydrocarbon groups containing 1 to 2 rings, including monocyclic or bicyclic groups. Cycloalkyl groups may comprise 3 or more carbon atoms in the ring and generally, according to this invention comprise from 3 to 10, more preferably from 3 to 8 carbon atoms still more preferably from 3 to 6 carbon atoms. The further rings of multi-ring cycloalkyls may be either fused, bridged and/or joined through one or more spiro atoms. Cycloalkyl groups may also be considered to be a subset of homocyclic rings discussed hereinafter. Examples of cycloalkyl groups include but are not limited to cyclopropyl, cyclobutyl, cyclopentyl, cyclohexyl, with cyclopropyl being particularly preferred. An “optionally substituted cycloalkyl” refers to a cycloalkyl having optionally one or more substituent(s) (for example 1 to 3 substituent(s), for example 1, 2 or 3 substituent(s)), selected from those defined above for substituted alkyl. When the suffix “ene” is used in conjunction with a cyclic group, this is intended to mean the cyclic group as defined herein having two single bonds as points of attachment to other groups.

The terms “heterocyclyl” or “heterocyclo” as used herein by itself or as part of another group refer to non-aromatic, fully saturated or partially unsaturated cyclic groups (for example, 3 to 7 member monocyclic, 7 to 11 member bicyclic, or containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valence allows. The rings of multi-ring heterocycles may be fused, bridged and/or joined through one or more spiro atoms. An optionally substituted heterocyclic refers to a heterocyclic having optionally one or more substituent(s) (for example 1 to 4 substituent(s), or for example 1, 2, 3 or 4 substituent(s)), selected from those defined above for substituted aryl.

Non limiting exemplary heterocyclic groups include aziridinyl, oxiranyl, thiiranyl, piperidinyl, azetidinyl, 2-imidazolinyl, pyrazolidinyl imidazolidinyl, isoxazolinyl, oxazolidinyl, isoxazolidinyl, thiazolidinyl, isothiazolidinyl, piperidinyl, succinimidyl, 3H-indolyl, indolinyl, isoindolinyl, 2H-pyrrolyl, 1-pyrrolinyl, 2-pyrrolinyl, 3-pyrrolinyl, pyrrolidinyl, 4H-quinolizinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, oxetanyl, thietanyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1,4-dithianyl, 1,3,5-trioxanyl, 1H-pyrrolizinyl, tetrahydro-1,1-dioxothiophenyl, N-formylpiperazinyl, and morpholin-4-yl.

The term “alkenyl” as used herein refers to an unsaturated hydrocarbyl group, which may be linear, branched or cyclic, comprising one or more carbon-carbon double bonds. Alkenyl groups thus comprise between 2 and 6 carbon atoms, preferably between 2 and 4 carbon atoms, still more preferably between 2 and 3 carbon atoms. Examples of alkenyl groups are ethenyl, 2-propenyl, 2-butenyl, 3-butenyl, 2-pentenyl and its isomers, 2-hexenyl and its isomers, 2,4-pentadienyl and the like. An optionally substituted alkenyl refers to an alkenyl having optionally one or more substituent(s) (for example 1, 2 or 3 substituent(s), or 1 to 2 substituent(s)), selected from those defined above for substituted alkyl.

The term “alkynyl” as used herein, similarly to alkenyl, refers to a class of monovalent unsaturated hydrocarbyl groups, wherein the unsaturation arises from the presence of one or more carbon-carbon triple bonds. Alkynyl groups typically, and preferably, have the same number of carbon atoms as described above in relation to alkenyl groups. Non limiting examples of alkynyl groups are ethynyl, 2-propynyl, 2-butynyl, 3-butynyl, 2-pentynyl and its isomers, 2-hexynyl and its isomers and the like. An optionally substituted alkynyl refers to an alkynyl having optionally one or more substituent(s) (for example 1 to 4 substituent(s), or 1 to 2 substituent(s)), selected from those defined above for substituted alkyl.

As previously described, the term “electroactive conjugated polymer” refers to conjugated polymers having the ability to undergo redox reaction when a voltage is applied to them. Thus, conjugated polymers as used in the invention can be polymers or copolymers based on heterocycle moiety as monomers, aniline and substituted aniline derivatives, cyclopentadiene and substituted cyclopentadiene derivatives, phenylene or substituted phenylene derivatives, pentafulvene and substituted pentafulvene derivatives, acetylene and substituted acetylene derivatives, indole and substituted indole derivatives, carbazole and substituted carbazole derivatives or compounds based on formula (I) or (II) wherein n is an integer, X is —NR¹—, O, S, PR², Si, Se, AsR³, BR⁴ wherein R and R′ which can be identical or not, linked or not, are alkyl, aryl, hydroxyl, alkoxy, wherein R¹, R², R³ and R⁴ are hydrogen, alkyl or aryl group and wherein A and A′ can be heterocycle, alkenyl, alkynyl or aromatic ring and wherein A and A′ can be identical or not.

In a preferred embodiment, the conjugated polymers are based on heterocycle moiety as monomers such as pyrrole and substituted pyrrole derivatives, furan and substituted furan derivatives, thiophene and substituted thiophene derivatives, phosphole and substituted phosphole derivatives, silole and substituted silole derivatives, arsole and substituted arsole derivatives, borole and substituted borole derivatives, selenole and substituted selenole derivatives or aniline and substituted aniline derivatives.

In a preferred embodiment, the conjugated polymers are based on pyrrole and substituted pyrrole derivatives.

Where the nanowires are hollow tubes formed from electroactive conjugated polymer, the spaces between the nanowires may be disposed with a matrix material. This is generally a layer of polymeric matrix 2. The polymeric matrix 2 may comprise a polymer chosen from the family of carbonic acid polyesters like bisphenol A polycarbonate, saturated polyesters like polyethyleneterephthalate or of polyimide. A role of the polymeric matrix 2 is to provide mechanical support to the nanowires.

The polymeric matrix 2 may have an average layer thickness before etching of 100 nm, 200 nm, 300 nm, 400 nm, 500 nm, 600 nm, 700 nm, 800 nm, 900 nm, 1000 nm (1 micron), 50 microns, 100 microns, 200 microns, 300 microns, 400 microns, 500 microns or a value in the range between any two of the aforementioned values. Preferably, the polymeric matrix 2 has an average layer thickness of between 100 nanometers and 100 microns, preferably between 100 nanometers and 50 microns and more preferably between 100 nanometers and 10 microns.

The nanowire array 15, 16, particularly incorporated in a stimulation electrode, will reduce nerve damage, and allows a response to be induced using less current or voltage. Generally, when foreign material such as an electrode is implanted in or around neural or other living tissues, a number of inflammatory and immunological reactions are triggered. Even if they are temporary reactions, some of these can be deleterious. For example, through oedema, a nerve can crush itself if a tight cylindrical electrode is implanted around it. The present invention allows the local diffusion of a drug that prevents oedema after implantation of tight electrodes without damaging the nerve. Through the elimination of a conducting layer between target and contact, such a tight electrode leaks current when used for stimulation. Nerve recording is also improved since less of the signal will be shunted in the intervening tissue. Furthermore, in the combination of stimulation and recording channels within the same device, there is less cross-talk between these channels.

The present invention can also be used to control the degree of fibrosis in the area of implantation. Tissue reaction around an implanted device can be drug-controlled but different drugs must sometimes be used on different parts of an implant. For example, locally delivered drugs may induce a reasonable degree of fibrosis in order to attach the device to surrounding tissues and prevent it from moving away from target. For easy removal, the selected form of tissue reaction is such that it does engulf the foreign material. On the other hand, in the case of electrodes for example, preventing the accumulation of scar tissue between the electric contacts and the target will likely improve the electrode efficiency. In addition, selective drugs could be used to avoid direct contact between cells and the metallic surface. A deposition of fibres insures lower impedance at the interface because the lipid cell membranes act as insulators.

The nanowire array 15, 16, incorporated in a stimulation electrode also reduces the contact impedance as its' capillary-like structure increases the real area to geometric area ratio of the electrode contacts. This is an efficient way to reduce electrode impedance because the metal to hydrated medium interface is by far the most significant component of that impedance. In addition, ions included in a polymer attached to the electrode contacts can deliver or recover charges at a low energy level and, therefore, replace the metal-ionic solution with a low impedance electron-to-ion conductance transformer.

The present invention also solves a problem with conventional electrode contacts of non-uniform current density at the surface; typically they have a much higher current density around the edges. A consequence is that current densities are dangerous for the surrounding tissue. Also electrode corrosion takes place at these high current density spots while much of the contact area is still not fully exploited. In one of the preferred embodiments of the present invention, a plurality of capillary-like wire densities or sizes is used in different regions of the contact area in order to compensate for the edge effect so that the current becomes uniform over the area and the overall current a contact safely delivers becomes much higher.

The nanowire array further provides an accurate local drug delivery system that is exquisitely controlled by current. The capillary-like area provided by the nanowire array increases the storage capacity availably for the therapeutic agent. The current-controlled release provides accuracy and to some extent, reversibility.

One embodiment of the invention is an electrode contact provided with a nanowire array 15, 16 in electrical connection with the electrode contact. Said electrode contact is able to release a therapeutic composition upon stimulation. A nanowire array according to the invention is disposed onto at least part of the electrode contact. Preferably the electrode contact of the invention is formed from a nanowire array 15, 16 where at least part of the electrode contact is the electrically conducting solid support 7 of the nanowire array 15, 16.

The electrode contact may be made from any suitable conducting material such as carbon, copper, titanium, gold, silver, platinum, palladium, bismuth, nickel or stainless steel. It is preferably made from a noble metal such as platinum or gold. It may be a metallic bounded contact. It will be configured according to known practices, for example, provided with one or more conducting wires at least partly insulated.

According to the invention, an electrode contact may be a circumneural contact, a small surface or a dot contact but are not limited to them. In a preferred embodiment, the electrode contact is a dot contact. The electrode contact may be recessed in non-conductive material, at the surface level or alternatively occupy entirely or partially a protruding shape such as a spike or any other geometrical volume.

Another embodiment of the invention is an electrical-stimulation or -recording electrode incorporating a nanowire array 15, 16 of the invention. The electrode comprises at least one electrode contact, wherein said electrically conducting solid support 7 of the array 15, 16 is formed from at least part of said electrode contact. Said electrode is able to release a therapeutic composition upon stimulation.

One embodiment of the invention is an electrode comprising 1, 2, 3, 4, 5, 6, 7, 8, 9, 10, 15, 20, 25, 30, 35, 40, 45, or 50 electrode contacts or a number between any two of the aforementioned values, wherein at least one contact is provided with a nanowire array according to the invention. Preferably, an electrode comprises between 1 and 50 electrode contacts, preferably between 1 and 20 and more preferably between 1 and 5 electrode contacts.

The electrode may be of any configuration, depending on the application and location of use. One embodiment of the invention is a self-sizing spiral cuff electrode comprising one or more electrode contacts as described above. In a preferred embodiment, the electrode is a self-sizing spiral cuff electrode as described, for example, in U.S. Pat. No. 4,602,624 which is incorporated herein by reference. A spiral cuff typically comprises two bonded flexible sheets, whereby one sheet has been stretched before bonding and the other not, or stretched to a lesser extent. The result is a drag force between the sheets that causes the assembly to curl. The amount of stretch determines the desired diameter: the greater the stretch, the smaller the diameter. The sheets will curl as a result of the drag force created between the layers.

One embodiment of the invention is a cuff electrode of the present invention, wherein the active biomolecule comprises a drug that prevents oedema. Said cuff electrode allows local release of drugs that allows implanting tight electrodes without damaging the nerve under electrical stimulation.

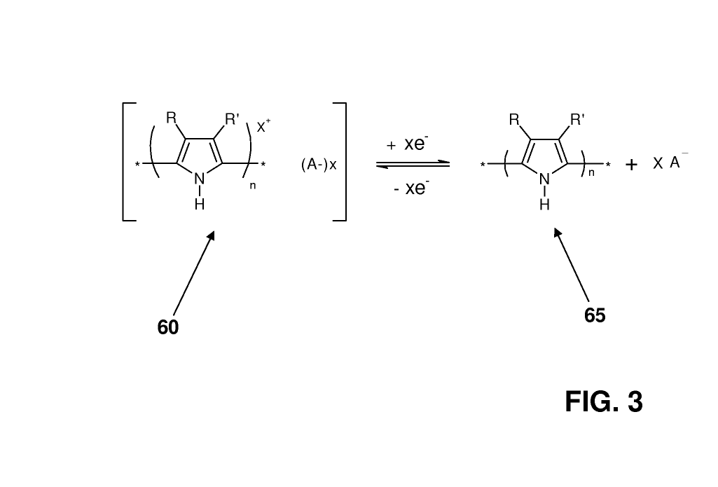

The electroactive conjugated polymers 4 have the ability to undergo reversible redox reaction and can be doped with hydrated ions. The doped polymer can be electrically switched between the oxidized and reduced state. The oxidized form is the conductive one while in reduced state polypyrrole is the neutral insulating form. The redox reaction modifies the shape of the polymer material. Swelling and shrinking of the polymer material occurs due to the incorporation or expulsion of hydrated ions. This movement of ions in and out of the electroactive conjugated polymer 4 constitutes the basic principle of drug release from an electroactive conjugated polymer. FIG. 3 depicts a redox process at the basis of drug release (A-), where polypyrrole in an oxidized conductive form 60 is shown converting to polypyrrole in a neutral insulating form 65. ‘A-’ represents hydrated ions and ‘x’ the oxidation state of pyrrole unit in polypyrrole.

According to one aspect of the present invention, the electroactive conjugated polymer 4 is doped or contains a therapeutic composition (drug) that is locally released upon further electrical stimulation. The therapeutic composition may comprise one or more bioactive molecules of interest including, for example, nutritional substances such as vitamins, antioxidants or minerals; active ingredients such as anticancer drugs, antipsychotic, antiparkinsonian agents, antiepileptic agents, antimigraine agents; nucleic acids such as nucleotides, oligonucleotides, antisense oligonucleotides, DNA, RNA and mRNA; amino acids and natural, synthetic and recombinant proteins, glycoproteins, polypeptides, peptides, enzymes; antibodies, hormones, cytokines and growth factors. Preferably, the therapeutic composition comprises one or more anti-inflammatory agents. More preferably, the therapeutic composition comprises one or more anti-TNF-alpha agents such as adalimumab, infliximab, etanercept, certolizumab pegol, and golimumab; one or more steroidal anti-inflammatory agents such as dexamethasone disodium; one or more non-steroidal anti-inflammatory agents such as aceclofenac, acemetacin, aspirin, celecoxib, dexibuprofen, dexketoprofen, diclofenac, diflunisal, etodolac, etoricoxib, fenbrufen, fenoprofen, flurbiprofen, ibuprofen, indomethacin, ketoprofen, ketorolac trometamol, lumiracoxib, mefanamic acid, meloxicam, nabumetone, naproxen, nimesulide, oxaprozin, parecoxib, phenylbutazone, piroxicam, proglumetacin, sulindac, tenoxicam, and tiaprofenic acid.

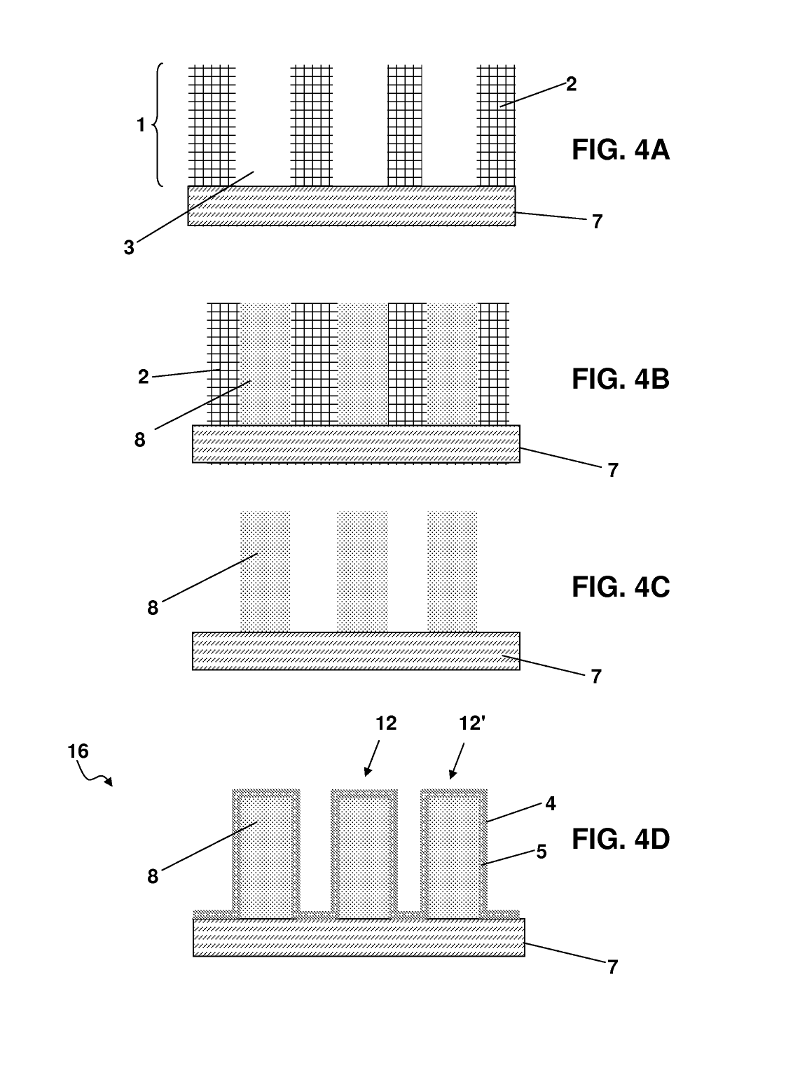

A first configuration of the nanowire array 16 is comprised of wires made from electroactive conjugated polymer coated conducting nanoscopic protrusions, whereby the coating is doped with therapeutic composition. FIGS. 4A to 4D show consecutive steps of a method for preparing the drug-eluting nanowire array depicted as a series of transverse cross-sections. In FIG. 4A, a nanoporous polymeric layer 1 disposed on an electrically conducing solid support 7 is formed by creating pores 3 in a layer of polymeric matrix 2 using, for example, track etching. In FIG. 4B, electrically conducting protrusions 8 are electrochemically grown within the pores 3 of the polymeric nanoporous layer 1. In FIG. 4C the nanoporous polymeric layer 1 is removed. In FIG. 4D, a layer of the electroactive conjugated polymer is electropolymerized onto the resulting electrically conducting protusions 8 in one step, in presence of therapeutic composition 5. The result is the nanowire array 15 of the second configuration, comprising a plurality of nanoscopic sized wires 12, 12′ of the invention.

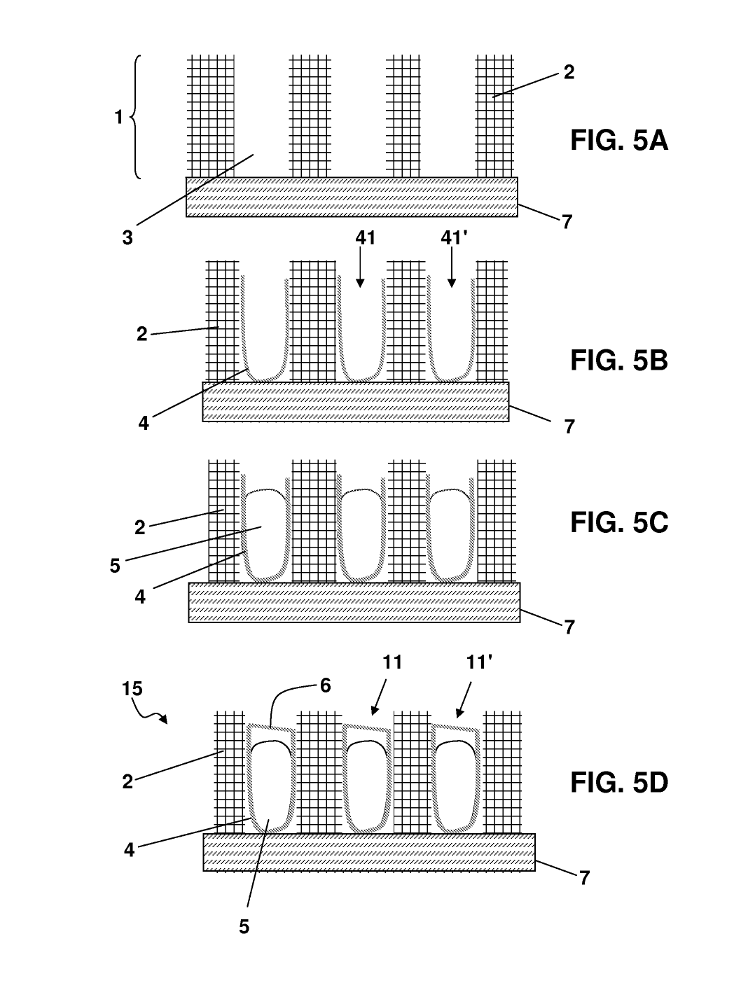

A second configuration of the nanowire array 15 is comprised of hollow wires made from electroactive conjugated polymer 4, the hollow in each wire containing therapeutic composition 5. FIGS. 5A to 5D show consecutive steps of a method for preparing the drug-eluting nanowire array, depicted as a series of transverse cross-sections. In FIG. 5A, a nanoporous polymeric layer 1 is formed by creating pores 3 in a layer of polymeric matrix 2 disposed on an electrically conducing solid support 7 using, for example, track etching. In FIG. 5B electroactive conjugated polymer 4 is electrochemically synthesized within the pores 3 of the nanoporous polymeric layer 1, resulting in hollow nanoscopic sized wires 41, 41′. In FIG. 5C, the hollow nanoscopic sized wires 41, 41′ receive the desired therapeutic composition 5. In FIG. 5D, a layer of electroactive conjugated polymer is electropolymerized across the open ends of the wires to form a cap 6 to retain the therapeutic composition 5 within. The result is the nanowire array 15 of the first configuration, comprising a plurality of nanoscopic sized wires 11, 11′ of the invention.

One aspect of the invention is a method for the preparation of a nanowire array that elutes a therapeutic composition comprising the steps of:

(a) depositing a layer of polymeric matrix 2 at onto at least part of an electrically conducing solid support 7,
(b) creating pores 3 in the layer polymeric matrix 2 by track-etching so forming a polymeric nanoporous layer 1,

### Either:

(c) electrodepositing an electrically conducting material 8 within the pores 3 of the polymeric nanoporous layer 1,
(d) dissolving the polymeric nanoporous layer 1 to form electrically conducting protrusions 8,
(e) electropolymerising an electroactive conjugated polymer 4 doped with therapeutic composition 5;
   or:
(C) electropolymerising an electroactive conjugated polymer 4 within the pores 3 of the polymeric nanoporous layer 1, so creating hollow nanoscopic sized wires 41, 41′
(D) applying the therapeutic composition 5 to the hollow of the wires 41, 41′,
(E) electropolymerising a layer of electroactive conjugated across the open end of the nanoscopic sized wires 41, 41′, to form a cap 6;
   so forming a nanowire array.

According to a first step (step (a)) of the method, a polymeric matrix 2 is deposited over at least part of an electrically conducing solid support to form a layer. This may be achieved by any suitable process such as spin coating. The polymeric matrix 2 can be made from carbonic acid polyesters like bisphenol A polycarbonate, saturated polyesters like polyethyleneterephthalate or of polyimide of a mixture thereof. In a preferred embodiment, the electrically conducing solid support is made of platinum and the polymeric matrix is made of polycarbonate. Polymeric matrix can be used as container or as barrier for the controlled drug release, but also as support for the synthesis of nanostructured electrodes.

The layer of polymeric matrix 2 and the subsequently formed polymeric nanoporous layer 1 may have an average thickness of 100 nm, 200 nm, 300 nm, 400 nm, 500 nm, 600 nm, 700 nm, 800 nm, 900 nm, 1000 nm (1 micron), 50 microns, 100 microns, 200 microns, 300 microns, 400 microns, 500 microns or a value in the range between any two of the aforementioned values. Preferably, the layer of polymeric matrix 2 and polymeric nanoporous layer 1 have an average thickness between 100 nanometers and 100 microns, preferably between 100 nanometers and 50 microns and more preferably between 100 nanometers and 10 microns.

According to a second step (step (b)) of the method, pores are created in the layer of polymeric matrix. This may be achieved by track etching techniques (Legras et al. EP1569742, EP 0262 187).

Track-etching technique relates to a technology of bombardment of polymeric films and coatings by energetic heavy ions such as Ar, Kr or Xe (produced for example in a cyclotron) followed by a selective chemical etching to make pores. Prior to etching, an irradiated layer of polymeric matrix is light sensitised with a UV or visible light source for 1 to 4 hours. This treatment remains a critical step in the process as it significantly influences the final pore size and shape of the track etched templates. In a preferred embodiment, the irradiated layer of polymeric matrix is light sensitised with UV light source. Chemical etching is then performed. This may be achieved using an aqueous solution of sodium hydroxide, preferably having a concentration between 0.5 mol/L and 2.0 mol/L. The chemical etching is performed at a temperature up to 70° C. and for a time between 15 minutes and 12 hours depending on the final requested pore size. In a preferred embodiment, the chemical etching is performed at a temperature around 70° C. for a time between 15 minutes and one hour.

For the last 15 years, the first generation track etching technology has been the basis for the commercial manufacture of porous polymer membranes used mainly for biomedical and separation applications. Since 1996 this technology has been significantly extended through a series of collaborative research projects to give new capabilities well into the true nano-range. More polymers can now be efficiently track-etched, control of pore shape and patterning of the zones where pores occur can now be achieved in membranes as well as in spin coatings on substrates such as silicon and glass. The nanoporous materials can also be “engineered” by filling the nanopores with metals, alloys or polymers to make in-situ nanowires or nanotubes; assembled into structures and components using nanofabrication, lamination and embossing techniques; and interfaced with electrical circuitry (Ferain et al. U.S. Pat. No. 6,861,006 and EP 1 242 170).

Capacities of the ‘first generation technology’ is mostly used to make porous polymer membranes, typically 10-20 μm thick, where the pores are randomly distributed and sizes are in the range 0.1 μm-10 μm. Polymers that are regularly ‘track-etched’ include polycarbonate (PC) and polyethylene terephthalate (PET).

The new ‘second generation technology’ (Ferain et al. US2006/000798 and EP 1 569 742) overcomes many of these limitations and offers advantages over the first generation products including:
true nanopores as small as 10 nm may be produced of controlled size and shape in a range of pore densities (number of pores/cm²);
the maximum operating temperature is now over 430° C. (previously 120° C.) thanks to a new patented method for track-etching polyimide polymers;
nanoporous spin-coated polymer layers, ˜200 nm-5 microns thick on glass, quartz and silicon, are now available for use in wafer or substrate based devices;
the geometry of the nanopores and nano-objects (aspect ratio or length/diameter) can be varied from 0.4 to over 2000 depending on whether spin-coated layers or freestanding films are used;
the nanoporous materials can be patterned using patented technology with nanopores localised into areas as small as 10 microns square.

The etching step provides a polymeric nanoporous layer 1 provided with a plurality of pores having a pore size of diameter of 10 nm, 50 nm, 100 nm, 200 nm, 300 nm, 400 nm, 500 nm, 600 nm, 700 nm, 800 nm, 900 nm, 1000 nm (1 micron), 2 microns, 3 microns, 4 microns, 5 microns, 6 microns, 7 microns, 8 microns, 9 microns, 10 microns or a value in the range between any two of the aforementioned values. Preferably a nanoscopic size wire 11, 11′, 12, 12′ has a diameter between 10 nm and 10 microns, preferably between 10 nanometers and 1 micron, more preferably between 10 and 500 nanometers. The pore size determines the diameter of the nanowires wires. The density of pores on the polymeric nanoporous layer 1 may be 5 pores/cm², 10 pores/cm², 10² pores/cm², 10³ pores/cm², 10⁴ pores/cm², 10⁵ pores/cm², 10⁶ pores/cm², 10⁷ pores/cm², 10⁸ pores/cm², 10⁹ pores/cm², and 10¹⁰ pores/cm² or a value in the range between any two of the aforementioned values. Preferably the pore density is between 10⁸ pores/cm² and 10⁹ pores/cm².

As mentioned above, the method may proceed (steps (c) to (e)) by electrodepositing an electrically conducting material within the pores, forming electrically conducting protrusions 8, and dissolving the polymeric matrix 2 and hence the polymeric nanoporous layer 1. Thus, in an embodiment of the invention, the step of electrodepositing an electrically conducting material within the pores, forming electrically conducting protrusions 8, and dissolving the polymeric nanoporous layer 1 is performed. The deposited electrically conducting material may be metallic. It may be made of noble metals such as platinum, gold, silver, palladium, bismuth, nickel. Alternatively, the electrically conducting material may be made from carbon. Preferably, the method according to the invention provides electrically conducting protrusions 8 made of platinum.

According to a preferred embodiment of the invention, the nanowires are electrodeposited by a chronoamperometry technique in aqueous medium. In chronoamperometry, the potential of the working electrode is stepped, and the resulting current from faradic processes occurring at the electrode is monitored as a function of time. By changing the chronoamperometry conditions, it is possible to control the length of the nanowires.

Once conducting protrusions 8 have been formed within the pores, the polymeric nanoporous layer is dissolved to reveal the structure of nanowires array. This step may be optimised to reduced the presence of any residue of polymeric nanoporous layer, thereby damaging the performance of electrodes.

Once conducting protrusions 8 have been formed, electroactive conjugated conjugated polymer 4 is electropolymerised thereon; the electropolymerisation is performed in the presence of the therapeutic composition as doping anions, so giving rise to nanoscopic sized wires and the nanowire array of the invention.

As mentioned above, the method may proceed alternatively (steps (C) to (E)) by electropolymerising the electroactive conjugated polymer within the pores of the polymeric nanoporous layer to form hollow wires, applying the therapeutic composition to the hollow and electropolymerising the conjugated polymer to close the open end of the nanoscopic sized hollow wires. The method also gives rise to nanoscopic sized wires and the nanowire array of the invention. The polymeric matrix may remain to support the structure of wires

According to one aspect of the invention, a nanoscopic size wire 11, 11′, 12, 12′ may have a diameter of 10 nm, 50 nm, 100 nm, 200 nm, 300 nm, 400 nm, 500 nm, 600 nm, 700 nm, 800 nm, 900 nm, 1000 nm (1 micron), 2 microns, 3 microns, 4 microns, 5 microns, 6 microns, 7 microns, 8 microns, 9 microns, 10 microns or a value in the range between any two of the aforementioned values. Preferably a nanoscopic size wire 11, 11′, 12, 12′ has a diameter between 10 nm and 10 microns, preferably between 10 nanometers and 1 micron, more preferably between 10 and 500 nanometers.

In another embodiment, nanoscopic size wire 11, 11′, 12, 12′ may have an aspect ratio (length/diameter) of 0.4, 1, 5, 10, 50, 100, 200, 300, 400, 500, 600, 700, 800, 900, 1000, 1200, 1400, 1600, 1800 2000 or a value in the range between any two of the aforementioned values. Preferably a nanoscopic size wire 11, 11′, 12, 12′ has an aspect ratio between 0.4 and 2000, preferably between 10 and 2000 and more preferably between 100 and 2000.

In another preferred embodiment, the method according to the electroactive conjugated polymer 4 is based on heterocycle moiety as described extensively elsewhere herein. Preferably the electroactive conjugated polymer 4 is a polypyrrole.

The so-prepared polypyrrole micro- or nano-structured modified electrode contacts are easily integrated into the cuff-electrode device for electrical neurostimulation by simply pasting them onto a medical device.

According to the invention, a polymeric nanoporous layer 1 used as template in the manufacture of the nanowire array. Said polymeric nanoporous layer 1 may be made of carbonic acid polyesters like bisphenol A polycarbonate, saturated polyesters like polyethyleneterephthalate or of polyimide. Preferably, the polymeric nanoporous layer is made of polycarbonate.

According to the invention, the polymeric nanoporous layer 1 used as template has an average thickness of 100 nm, 200 nm, 300 nm, 400 nm, 500 nm, 600 nm, 700 nm, 800 nm, 900 nm, 1000 nm (1 micron), 50 microns, 100 microns, 200 microns, 300 microns, 400 microns, 500 microns or a value in the range between any two of the aforementioned values. Preferably, the polymeric nanoporous layer 1 has an average thickness between 100 nanometers and 100 microns, preferably between 100 nanometers and 50 microns and more preferably between 100 nanometers and 10 micron.

The density (number of nanowires/cm²) of nanoscopic size wire 11, 11′, 12, 12′ present in a nanowire array may be dependent of the pore density of the polymeric nanoporous layer and partly dependent on the nanoscopic size wire 11, 11′, 12, 12′ (nanowire) diameter. The density of nanowires may be 5 nanowires/cm², 10 nanowires/cm², 10² nanowires/cm², 10³ nanowires/cm², 10⁴ nanowires/cm², 10⁵ nanowires/cm², 10⁶ nanowires/cm², 10⁷ nanowires/cm², 10⁸ nanowires/cm², 10⁹ nanowires/cm², and 10¹⁰ nanowires/cm² or a value in the range between any two of the aforementioned values. Preferably the nanowires density is between 10⁵ pores/cm² to 10⁹ pores/cm², preferably between 10⁸ and 10⁹ pores/cm².

A method for preparing a drug eluting electrode contact according to the invention may follow the steps of preparing a nanowire array described herein, wherein the electrically conducing solid support 7 is at least part of an electrode contact. The electrode contact can be incorporated into stimulation or recording electrodes depending on the medical application (see below). For example, it may be used to form an electrode suitable for vagus nerve stimulation, deep brain stimulation, cochlear stimulation, brain stimulation.

The present invention may also be used to deliver exquisitely-controlled quantities of therapeutic composition to a region of implantation, for example, to control delivery of a chemotherapy agent or of a chemotherapy sensitizing agent.

An electrode contact of the present invention may be incorporated into a cuff electrode as described above. Cuff manufacturing technique and general description of a self-sizing spiral cuff electrode (Naples et al. patent number: U.S. Pat. No. 4,602,624 “Implantable cuff, method of manufacture, and method of installation”; PhD Thesis Romero E. and Thil M.-A. School of medicine, Universite Catholique de Louvain, Brussels, Belgium respectively in 2001 and 2006).

In a preferred embodiment, the spiral cuff comprises two bonded flexible sheets, whereby one sheet has been stretched before bonding and the other not, or stretched to a lesser extent. The result is a drag force between the sheets that causes the assembly to curl. The amount of stretch is determined by the desired diameter of the cuff: the more the stretch, the smaller the diameter.

Thus, one embodiment of the invention relates to a method for preparing a spiral cuff electrode having an inward facing surface disposed with an electrode contact, and an outward facing surface, said method further comprising the steps of:
bonding one surface of an unstretched flexible sheet to one surface of a stretched flexible sheet wherein the stretched flexible sheet has been stretched in one direction prior to bonding, and
providing an electrode contact as defined above, located on or in the inward facing surface, so forming a spiral cuff electrode.

According to one aspect of the invention, the flexible sheets are made from silicone elastomer e.g. silicone rubber.

The basic technique behind the fabrication of a spiral cuff is subjecting two flexible bonded sheets each to a different strain in a specific direction. The sheets will curl as a result of the drag force created between the layers.

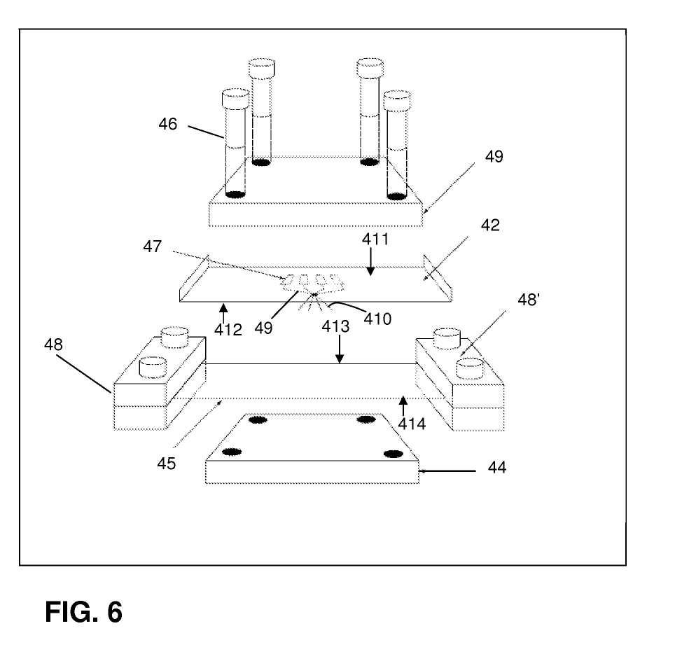

The spiral cuff is manufactured by any suitable method in the art. According to one aspect of the invention, the spiral cuff is prepared by applying a bonding (adhesive) substance such as unpolymerised adhesive silicone layer to one surface of a stretched sheet 45 (see FIG. 6). Subsequently, an unstretched sheet 42 is placed in contact with the adhesive side of the stretched sheet, and the assembly is compressed to a determined and constant thickness. The thickness of the assembly may be 20 μm, 30 μm, 40 μm, 50 μm, 60 μm, 70 μm, 80 μm, 90 μm, 100 μm, 110 μm, 120 μm, 130 μm, 140 μm, 150 μm or a value in the range between any two of the aforementioned values, preferably between 70 and 90 μm.

After polymerisation, a central area of the assembly, where tension lines are more parallel, is selected for trimming the cuff. The result is a flexible sheet which naturally coils into a tubular spiral. This is due to the remaining tension in the inner layer pulling by friction on the unstretched layer. It forces the exterior sheet to follow the inner one and the effect of curling is obtained.

The number of wraps is determined in function of the target peripheral nerve. The number of wraps may be 1 to 3.5. In general, two and a half wraps assure a steady inner diameter of the cuff. Different recording and stimulating geometries can be created by correspondingly placing metallic contacts between the two sheets. Circumneural contacts, dot contacts and elongated patches along the nerve axis are the major shapes, but any contact arrangement with different sizes and shapes is possible.

FIG. 6 shows a view of the construction of a cuff electrode comprising four dot electrode contacts. A rectangle of expandable flexible sheet is clamped by two lateral clamps 48, 48′ and stretched linearly resulting in a stretched sheet 45 preferably having a thickness of around 80 μm. A rectangle of unstretched sheet 42 is aligned parallel with stretched sheet 45. Said unstretched sheet 42 is disposed with a set 47 of four dot electrode contacts formed of platinum foil (e.g. 25 μm thickness). Said contacts are disposed on the adhesive side of the unstretched sheet 42.

The adhesive side 413 of the stretched 45 sheet or the adhesive side 412 of the unstretched sheet 42 refers to the side of the sheet that comes into contact with adhesive and which is bonded to the other surface. It may the side onto which adhesive is applied. Alternatively, or it may be the side which comes into contact with the adhesive applied to the sheet, for example, when adhesive is applied only to one sheet.

The contacts are spot welded to an insulated connecting wire 49, which has been stripped at its tip to make the connection. The wire is made from any suitably conducting material such as copper, titanium, gold, silver, platinum, stainless steel; preferably it is made from stainless steel. Alternatively, the contacts and wire can be replaced by direct metallization of tracks on the silicone rubber. The unstretched sheet 42 is wrapped around an upper plate 49. It may be slightly stretched before wrapping around the upper plate 42 to obtain a smooth surface, essentially devoid of wrinkles. Adhesive e.g. unpolymerised silicone is applied to one sheet, preferably to the stretched sheet. By avoiding wrinkles, there will be an homogeneous diffusion of the adhesive.

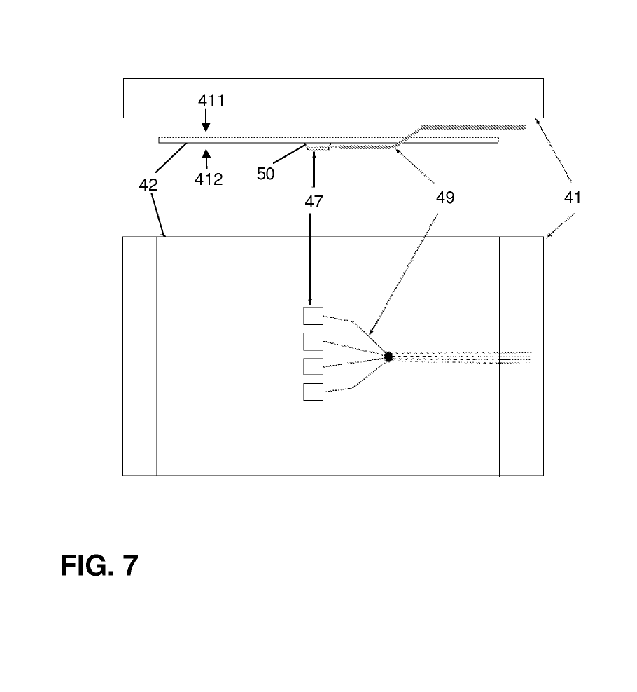

The electrode wires 49 should preferably by secured so that they avoid substantial movement between the two sheets. This may be achieved in part by allowing the wires to pass through the unstretched sheet 42, from the adhesive side 412 to the non-adhesive side 413 (FIG. 7). Preferably the wires 49 pass through the same opening.

It is noted that the non-adhesive side 411 of the unstretched sheet 42 will form the outward facing surface of the spiral cuff, while the non-adhesive side 414 of the stretched sheet 45 will form the inward facing surface of the spiral cuff.

Referring back to FIG. 6, a layer of adhesive, preferably unpolymerised silicone elastomer is applied to the adhesive surface 413 of the stretched sheet 45. The unstretched sheet 42 with the bonded contacts 47 is then placed in contact with said unpolymerised silicone elastomer. The composite so formed is squeezed to a thickness of around 250 μm using spacers.

As understood by the skilled person, each plate 49 of the press must have a perfectly plane surface to compress the cuff to a uniform thickness. Further, the lateral clamps 48, 48′ should be in the same condition to allow stretching of the sheet with no change in tension during the gluing process. After the gluing step and after cooling, an unsharped screw, placed near the frontal border is used to lift up the two plates.

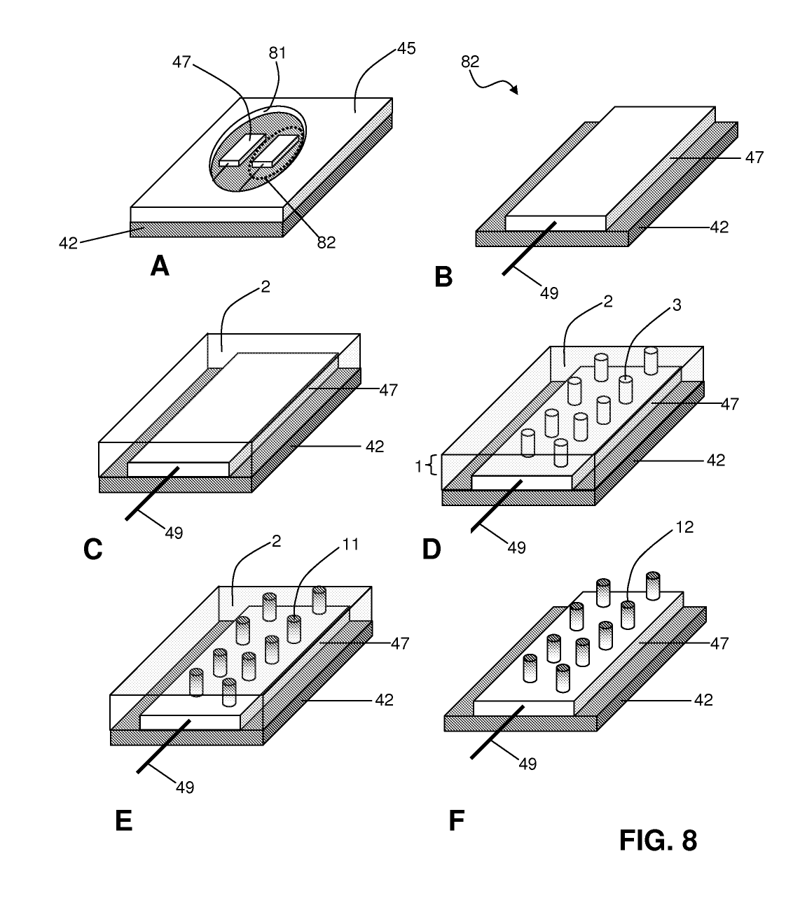

Referring to FIG. 8 after the gluing, a window 81 (FIG. 8A) is cut into the inward facing surface of the cuff to expose the metal contact to the cuff inside. The cut will therefore be applied to the previously stretched sheet 45. Laser cutting provides the best results. The window is preferably circular, but may as well by rectangular, oval, or other shape, including an irregular shape. A circular recession of the contact window creates a more uniform density current field across the surface of the electrode. This recess shape thereby decreases corrosion at the edges of electrode contacts. The strain profile along the bonded bi-layer is considered constant. Nevertheless, because the stretched sheet pulls in the middle (Poisson effect) this is correct only in the middle of the sheet where tension lines are parallel.

After cutting a window 81, a nanowire array of the invention is formed on the electrode contacts. The steps are depicted in FIGS. 8B to 8F which figures show the process applied to a single electrode 82 indicated in FIG. 8A. The exposed electrode contact 47, attached to the unstretched sheet 42 (FIG. 8B) is coated with a layer of polymeric matrix 2 (FIG. 8C) as described earlier. Using the preferred technique of track etching, a plurality of pores 3 is made into the layer polymeric matrix (FIG. 8D) so forming a polymeric nanoporous layer 1. Subsequently, the pores 3 are either used to form hollow nanoscopic sized wires from electroactive conjugated polymer, containing therapeutic composition (FIG. 8E) or used to form conductive (e.g. metal) nanosized protrusions coated with electroactive conjugated polymer doped with therapeutic composition (FIG. 8F).

The cuff is subsequently cut and trimmed according to desired dimensions. The length of the unrolled cuff varies according to the target nerve, the type of electrode and the particular application. For a diameter of about 2.5 mm, provided two full wraps will be around the nerve trunk, about 27 mm are necessary. Trimming that provides a bevelled edge is preferred to avoid sharp borders between the cuff and the nerve. Preferably, the cuff is trimmed using a 45° angled cut to give said bevelled edge.

The desired curling properties of the cuff can be achieved by applying known principles regarding the relationship between the stretch and the desired diameter, as, for example, derived by Naples et al. (Naples et al “A spiral nerve cuff electrode for peripheral nerve stimulation”. IEEE Trans Biomed Eng, 1988; 35(11): 905-916; U.S. Pat. No. 4,602,624).

For some applications a flat electrode shape is require. Such an electrode can be constructed exactly as described above except that no stretching will be applied with the consequence that the electrode will not curl.

The vagus nerve stimulation represents an important example in the application domain of the present invention. It is used in the treatment of conditions such as epilepsy, obesity, depression, anxiety disorders and other psychiatric diseases, migraines, fibromyalgia, Alzheimer's disease and Parkinson's disease. Just as for other functional nerve stimulation applications, it will directly benefit from more stable electrodes (more reliable stimulation) characterized by lower impedances (lower power consumption) and a more uniform current density (less electrode erosion). All these advantages will converge to allow the construction of high density electrodes through the smaller contacts, smaller implanted devices and the lower power consumption.

Refractory cases of epilepsy, pain, depression and other psychiatric diseases, Alzheimer's disease and especially Parkinson's disease, as well as various movement disorders can be efficiently treated with a multi-contact rod electrode inserted into the brain itself. The electrode shape is the reverse of a cuff electrode, now having the silicone rubber or other support material in the axial position and the neural tissue around it. Again, local control by additional drug delivery has the potential to increase significantly the efficiency of Deep Brain Stimulation.

Cochlear implants are already very popular but could still gain much efficiency by the higher resolution and reduced power waste made possible with this invention. Similarly, implants for incontinence, impotentia, motor palsy and the visual prosthesis, for example, use cuff electrodes and would therefore benefit from the same advantages as the vagus nerve stimulation. The possibility to place a large number of small contacts on the same device is one of the results of the reduced interface impedance, better current distribution and lower current waste as already mentioned. This will benefit resolution and thus also the possibility to stimulate selectively small subsets of nervous tissue.

This new field of development aims at interfacing electronic devices to the human brain with a bi-directional information exchange. Such a device should not only transmit signals from a device to the nervous system as is most often done in the prostheses above but also from the brain to the device. Such systems are needed by quadriplegic or locked-in patients in order to give them communication means and a control on their surrounding. Other applications involve direct brain to machine (often computer) interfaces in the hope to augment human capability. In animals, it can be used to control their behaviour.

Precise and adaptable local drug delivery is a major advantage in oncology for two reasons. The first one is that drugs used to kill a tumour are often poorly selective. It is therefore essential to deliver them locally and at the right dosage, enough to kill the cancer cells but not enough to induce collateral damage by diffusion. The second aspect concerns sensitisation drugs. These are drugs that increase the sensitivity of the cancer cells to another form of treatment, being heating or ionising radiation for example. The sensitising drug must be delivered locally at the right time for the main treatment to work optimally. A drug releasing electrode as described here is well adapted to such needs.

The pharmacological control of the local inflammatory reaction represents one of the main challenges in order to improve the efficacy of electrodes as explained above. However, such a local control can be applied to many focal inflammatory diseases through the controlled local drug and agent delivery feature being implemented in appropriately shaped silicone sheets or other support materials. Some candidate agents working as mediators of inflammation have been identified. Among them, TNF-alpha plays an important role in this paradigm. This factor appears to be an excellent target in order to improve the efficacy of implanted electrodes. It is indeed involved in the epineurial inflammation, the earliest event occurring after electrode implantation and it has pro-fibrotic action. Any attempt to block the production, the processing or the biological activity of TNF-alpha has already been proved to reduce pain-related behavior in rodents, as well as the local epineurial fibrotic reaction when administered systematically. It makes therefore sense to deliver anti TNF-alpha drugs locally in order to reduce systemic adverse effects, and also the cost related to type of therapy. In addition, the possibility of anti TNF-alpha local delivery will offer the opportunity to control some central and peripheral refractory neuropathic pains such as those observed in tetra or paraplegic patients, in diabetic patients or after herpetic infection. By extension, an improved local delivery of anti-inflammatory drugs will also find application for the treatment of inflammatory disease such as rheumatoid arthritis, patients with Crohn's disease, psoriatric arthritis, ankylosing spondylitis. Since atherosclerosis also results from inflammatory processes occurring in the vessel layer, one could expect an improvement in the plaque stabilization by reducing the local inflammation responsible for the onset of instable plaques through the local delivery of anti-inflammatory substances.

The nanowire array may be incorporated into a high resolution (spatial) electrode for use as a visual prosthesis, for example, where therapeutic agent can be selectively delivered precisely to a selected location. A visual prosthesis for blind Retinitis Pigmentosa patients is based the local delivery of neurotransmitters on the retina at selected points under the influence of light. This is presently achieved by the use of cage molecules such as fullerenes but is impeded by chemical toxicity and the required light levels to open the cages. Others explore the possibilities of micro-fluidic devices which are still too bulky for a realistic application. The present invention may be implanted on the retina while carrying on its back microscopic photosensitive elements each controlling the local delivery of a neurotransmitter that would activate the corresponding ganglion cells and recreate the normal image.

### Some Embodiments of the Invention

One embodiment of the invention is a nanowire array (15, 16) for electrically-controlled elution of a therapeutic composition (5) comprising a plurality of nanoscopic-sized wires (11, 11′, 12, 12′), nanowires, attached to an electrically conducting solid support (7), said nanowires formed from electroactive conjugated polymer (4) containing or doped with said therapeutic composition (5).

Another embodiment of the invention is a nanowire array (15, 16) for electrically-controlled elution of a therapeutic composition (5) comprising a plurality of nanoscopic-sized wires (12, 12′), nanowires, attached to an electrically conducting solid support (7), said nanowires formed from electroactive conjugated polymer (4) containing or doped with said therapeutic composition (5) coated over a plurality of nanoscopic sized electrically conducting protrusions (8).

Another embodiment of the invention is a nanowire array (15, 16) as described above, wherein a nanowire (11, 11′, 12, 12′) of said array (15, 16) has an elongate shape having a width between 10 nm and 10 microns.

Another embodiment of the invention is a nanowire array (15, 16) as described above, wherein a nanowire (11, 11′, 12, 12′), of said array (15, 16) has an aspect ratio (length/width) between 0.4 and 2000.

Another embodiment of the invention is a nanowire array (15, 16) as described above, wherein a nanowire (11, 11′, 12, 12′) is oriented essentially perpendicular to a surface of the electrically conducting solid support (7).

Another embodiment of the invention is a nanowire array (15, 16) as described above, wherein said electroactive conjugated polymer (4) is formed from monomers of any of pyrrole or substituted pyrrole derivatives, aniline or substituted aniline furan or substituted furan derivatives, thiophene or substituted thiophene derivatives, phosphole or substituted phosphole derivatives, silole or substituted silole derivatives, arsole or substituted arsole derivatives, borole or substituted borole derivatives, selenole, substituted selenole derivatives or aniline and substituted aniline derivatives.

Another embodiment of the invention is a nanowire array (15, 16) as described above, wherein the electroactive conjugated polymer (4) is a polymer comprising a compound of formula (I) or (II)

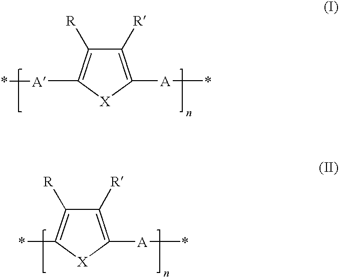

wherein
n is an integer greater than or equal to 3,
X is selected from the group consisting of —NR¹—, O, S, PR², SiR⁵R⁶, Se, AsR³, BR⁴ wherein R¹, R², R³, R⁴, R⁵ and R⁶ are independently selected from the group consisting of hydrogen, alkyl or aryl group,
R and R′ are independently selected from the group consisting of, alkyl, aryl, hydroxyl, alkoxy or R and R′ together with the carbon atoms to which they are attached form a ring selected from aryl, heteroaryl, cycloalkyl, heterocyclyl, and
A and A′ are independently selected from the group consisting of heterocyclyl, alkenyl, alkynyl or aromatic ring.

Another embodiment of the invention is a nanowire array (15, 16) as described above, wherein said electroactive conjugated polymer (4) is a polypyrrole.

Another embodiment of the invention is a nanowire array (15, 16) as described above, wherein said electroactive conjugated polymer (4) is formed as a plurality of hollow nanoscopic wires (11, 11′) which contain said therapeutic composition (5), and the spaces between the wires are disposed with a layer of polymeric matrix (2).

Another embodiment of the invention is a nanowire array (15, 16) as described above, wherein said polymeric matrix (2) is made from polycarbonate, polyethyleneterephthalate or polyimide.

Another embodiment of the invention is a nanowire array (15, 16) as described above, wherein said layer of polymeric matrix (2) has an average thickness of between 100 nanometers and 100 microns.

Another embodiment of the invention is a nanowire array (15, 16) as described above, wherein said electroactive conjugated polymer (4) doped with said therapeutic composition (5) and coated over a plurality of nanoscopic sized electrically conducting protrusions (8), forms the plurality of nanoscopic wires (12, 12′).

Another embodiment of the invention is a nanowire array (15, 16) as described above, wherein said nanoscopic sized electrically conducting protrusions (8) are formed from copper, titanium, gold, silver, platinum, palladium, bismuth, or nickel.

Another embodiment of the invention is a nanowire array (15, 16) as described above, where said nanoscopic sized electrically conducting protrusions (8) are of suitable size and shape to provide, after coating with electroactive conjugated polymer (4) doped with said therapeutic composition (5), a nanowire (12, 12′) having dimensions as defined above.

Another embodiment of the invention is a nanowire array (15, 16) as described above, wherein said electrically conducting solid support (7) is made from any of copper, titanium, gold, silver, platinum, palladium, bismuth, nickel, stainless steel, preferably platinum.

Another embodiment of the invention is a nanowire array (15, 16) as described above, wherein said therapeutic composition (5) comprises one or more nutritional substances including vitamins, antioxidants or minerals.

Another embodiment of the invention is a nanowire array (15, 16) as described above, wherein said therapeutic composition comprises (5) at least one TNF-alpha inhibitor.

Another embodiment of the invention is a nanowire array (15, 16) as described above, wherein said TNF-alpha inhibitor is any of adalimumab, infliximab, etanercept, certolizumab pegol, or golimumab.

Another embodiment of the invention is a nanowire array (15, 16) as described above, wherein said therapeutic composition (5) comprises at least one anti-inflammatory agent.

Another embodiment of the invention is a nanowire array (15, 16) as described above, wherein said anti-inflammatory agent is any of dexamethasone disodium, aceclofenac, acemetacin, aspirin, celecoxib, dexibuprofen, dexketoprofen, diclofenac, diflunisal, etodolac, etoricoxib, fenbrufen, fenoprofen, flurbiprofen, ibuprofen, indomethacin, ketoprofen, ketorolac trometamol, lumiracoxib, mefanamic acid, meloxicam, nabumetone, naproxen, nimesulide, oxaprozin, parecoxib, phenylbutazone, piroxicam, proglumetacin, sulindac, tenoxicam or tiaprofenic acid.

Another embodiment of the invention is a nanowire array (15, 16) as described above, wherein said therapeutic composition (5) comprises an active compound that is an anticancer drug, antipsychotic, antiparkinsonians agent, antiepileptic agent, or antimigraine agent.

Another embodiment of the invention is a nanowire array (15, 16) as described above, wherein said therapeutic composition (5) comprises nucleic acids such as nucleotides, oligonucleotides, antisense oligonucleotides, DNA, RNA and mRNA; amino acids and natural, synthetic and recombinant proteins, glycoproteins, polypeptides, peptides, enzymes, antibodies, hormones, cytokines and growth factors.

Another embodiment of the invention is a nanowire array (15, 16) as described above, configured such that the ratio of real area to geometric area is greatest at the centre of the array, allowing compensation for non-uniform current density at the array surface.

Another embodiment of the invention is an electrode contact wherein the electrically conducting solid support (7) of the array (15, 16) as defined above is formed from at least part of said electrical contact.

Another embodiment of the invention is an electrical-stimulation or -recording electrode incorporating an electrode contact as defined above.

Another embodiment of the invention is an electrode as defined above, configured as a cuff electrode.

Another embodiment of the invention is an electrode as defined above, configured as a vagus nerve stimulation and/or recording electrode.

Another embodiment of the invention is an electrode as defined above, configured as a peripheral nerve stimulation and/or recording electrode.

Another embodiment of the invention is an electrode as defined above, configured as a deep brain stimulation and/or electrode.

Another embodiment of the invention is an electrode as defined above, wherein said electrode is incorporated into a cochlear implant.

Another embodiment of the invention is an electrode as defined above, configured as a brain stimulation and recording electrode.

Another embodiment of the invention is an electrode as defined above, configured as a tumour implantable device.

Another embodiment of the invention is an electrode as defined above, configured as a subcutaneously implantable device.

Another embodiment of the invention is an electrode as defined above, incorporated into a visual prosthesis.

Another embodiment of the invention is a method for the preparation of a nanowire array (15, 16) for eluting a therapeutic composition (5) comprising the steps of:

(a) depositing a layer of polymeric matrix (2) onto at least part of an electrically conducting solid support (7),
(b) creating pores (3) in the layer of polymeric matrix (2) by track-etching so forming a polymeric nanoporous layer (1), either:
(c) electropolymerising an electroactive conjugated polymer (4) within the pores (3) of the polymeric nanoporous layer (1), so creating hollow nanoscopic sized wires (41, 41′).
(d) applying the therapeutic composition (5) to the hollow of the wires (41, 41′),
(e) electropolymerising a layer of electroactive conjugated across the open end of the nanoscopic sized wires (41, 41′), to form a cap (6);
   or:
(C) electrodepositing an electrically conducting material within the pores (3) the polymeric nanoporous layer (1),
(D) dissolving the polymeric nanoporous layer (1) to form electrically conducting protrusions (8),
(E) electropolymerising onto said protrusions (8) an electroactive conjugated polymer (4) doped with therapeutic composition (5);
   so forming a nanowire array (15, 16).

Another embodiment of the invention is a method for the preparation of a nanowire array (15, 16) for eluting a therapeutic composition (5) comprising the steps of:

(a) depositing a layer of polymeric matrix (2) onto at least part of an electrically conducting solid support (7),
(b) creating pores (3) in the layer of polymeric matrix (2) by track-etching so forming a polymeric nanoporous layer (l),
(c) electrodepositing an electrically conducting material within the pores (3) the polymeric nanoporous layer (l),
(d) dissolving the polymeric nanoporous layer (1) to form electrically conducting protrusions (8), and
(e) electropolymerising onto said protrusions (8) an electroactive conjugated polymer (4) doped with therapeutic composition (5);
so forming a nanowire array (15, 16).

Another embodiment of the invention is a method as described above, wherein
the polymeric matrix (2) is as defined above,
the electrically conducing solid support (7) is as defined above,
the electroactive conjugated polymer (4) is as defined above,
the therapeutic composition (5) is as defined above, and
conducting protrusions (8) is as defined above.

Another embodiment of the invention is a method for preparing an electrode contact comprising the method for the preparation of a nanowire array as described above, where in the electrically conducing solid support (7) is formed by at least part of a contact of the electrode.

Another embodiment of the invention is a method for preparing a spiral cuff electrode having an inward facing surface disposed with an electrode contact, and an outward facing surface, said method further comprising the steps of:
bonding one surface of an unstretched flexible sheet to one surface of a second flexible sheet wherein the second flexible sheet has been stretched or not in one direction prior to bonding, and
providing an electrode contact using the method as defined above located on or in the inward facing surface, so forming a spiral cuff electrode or a flat sheet electrode.

Another embodiment of the invention is a method for preparing a spiral cuff electrode as described above, wherein the flexible sheets are made from a silicone elastomer, preferably silicone rubber.

Another embodiment of the invention is a method for preparing a spiral cuff electrode as described above, wherein the contact electrode is provided between the bonded sheets, and is exposed by an opening in the stretched flexible sheet.

### EXAMPLES

The invention is illustrated by the following non-limiting examples

### 1. Preparation of a Nanowire Array

A polymeric matrix of polycarbonate film was deposited onto an electrically conducting support of metallic gold. Cylindrical pores of nanoscale dimensions were formed in the polycarbonate by a process of track-etching. The density and diameter of pores varied depending on the experimental conditions.

Next, electrically conducting protrusions of platinum were formed in the pores of the polycarbonate film by an electroplating process. The sample was placed in an electroplating bath disposed with three electrodes. The aqueous electroplating solution comprised 0.01 M Na₂PtCl₆.6H₂O and 0.5M H₂SO₄. The polycarbonate film, metallised on one side with metallic gold, was used as the working electrode. The counter-electrode was a platinum electrode, and the reference electrode was an Ag/AgCl electrode. Electroplating of platinum was performed by chronoamperometry at room temperature and at a potential of 0 V compared with the Ag/AgCl electrode.

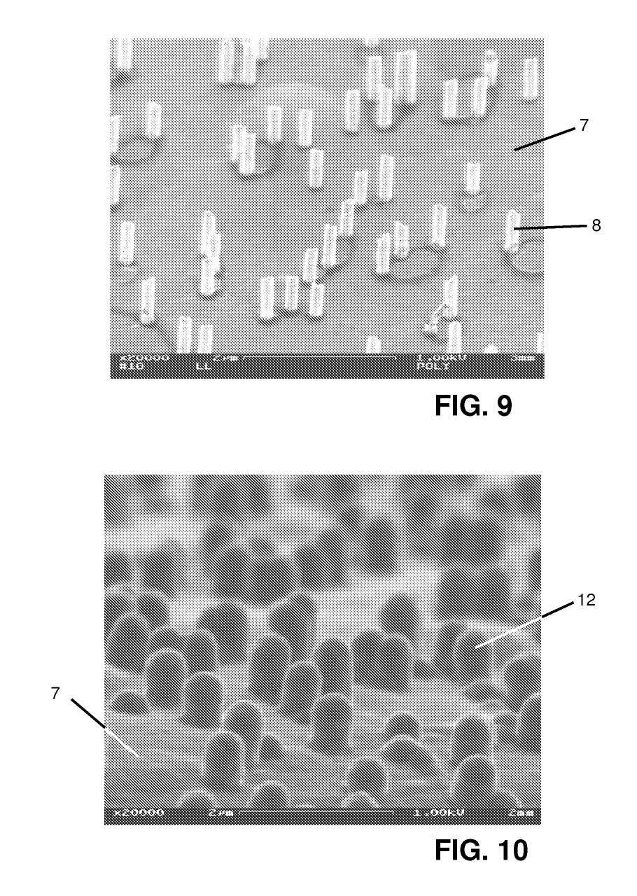

After growth of the metallic protrusions in the pores of the polycarbonate layer, the layer was dissolved to obtain the array of platinum nanosized protrusions. In a first stage of the dissolution process, the sample was immersed four or five times in dichloromethane for between 5 and 30 seconds to dissolve the majority of the polycarbonate layer. In a second stage, to dissolve the polycarbonate layer in stubborn areas (e.g. between the metallic surface and nanowires), longer cycles (e.g. 15 minutes) of dissolution with dichloromethane were performed. The operation was repeated four times, with a dichloromethane rinse between each cycle. Finally, remaining polymer was hydrolysed using a dilute basic solution i.e. 0.1 M NaOH; the sample was incubated twice for 5 minutes in the basic solution, then rinsed in dichloromethane. The dissolved polycarbonate layer revealed the structure of the nano-sized protrusions 8 on an electrically conducting solid support 7 as shown in FIG. 9.

### 2. Electropolymerisation of Electroactive Conjugated Polymer

An electroactive conjugated polymer that comprises pyrrole doped with therapeutic composition (dexamethasone) was deposited onto the metallic protrusions using an electropolymerisation technique. This oxidative polymerization was accompanied by the incorporation of molecules of interest (dexamethasone) to ensure the neutrality of the synthesised coating. Dexamethasone is a synthetic glucocorticoid hormone that has effects on reducing inflammation of the central nervous system and an immunosuppressive effect. This is currently one of the most powerful anti-inflammatory chemicals. The solution for the synthesis of polypyrrole/dexamethasone coating contained the pyrrole monomer at a concentration of 0.1 M and dexamethasone at a concentration of 0.025 M. The electropolymerisation was effected by chronoamperometry at a potential of 0.8 V compared with an Ag/AgCl electrode. Samples of different thicknesses were synthesized on nanostructured electrodes by adjusting the deposition time (see results below). At the end of the deposition, the sample was flushed to remove ions non-specifically adsorbed on the surface of the electroactive conjugated polymer coating. FIG. 10 shows a nanowire array of the invention comprising a plurality of nanoscopic-sized wires 12 that are nano-sized platinum protrusion coated with of polypyrrole/dexamethasone (PPy/DEX) disposed on the electrically conducting solid support 7.

### 3. Determination by UV-Vis of the Amount of Dexamethasone Released

Once the nanowire arrays were manufactured, their performances were evaluated. This entailed passing an electrical signal as a variable potential through each array in turn, and analyzing the effect of the signal on the amount of active ingredient released into the environment of the array and from the coating.

The therapeutic composition was released by cyclovoltametric scanning, the current passing being alternately cationic and anionic, leading to reactions of reduction and oxidation in the polypyrrole coating. The reduction involves the release of dexamethasone ions from the coating, while oxidation lead to the insertion of ions smaller from the buffer where experiments were conducted.

### Calibration

A control PBS solution formed from 20 mM NaH₂PO₄+20 mm Na₂HPO₄+150 mM NaCl and without any trace of dexamethasone was measured by UV-vis to determine the absorbance baseline. To determine the calibration curve, a series of solutions of different concentrations of dexamethasone was prepared and the absorbance of the different solutions at 242 nm measured and a relationship between concentration of dexamethasone (C) and UV-vis (A) was established: A=0.0196 C or C=51 A.

### Release of Dexamethasone

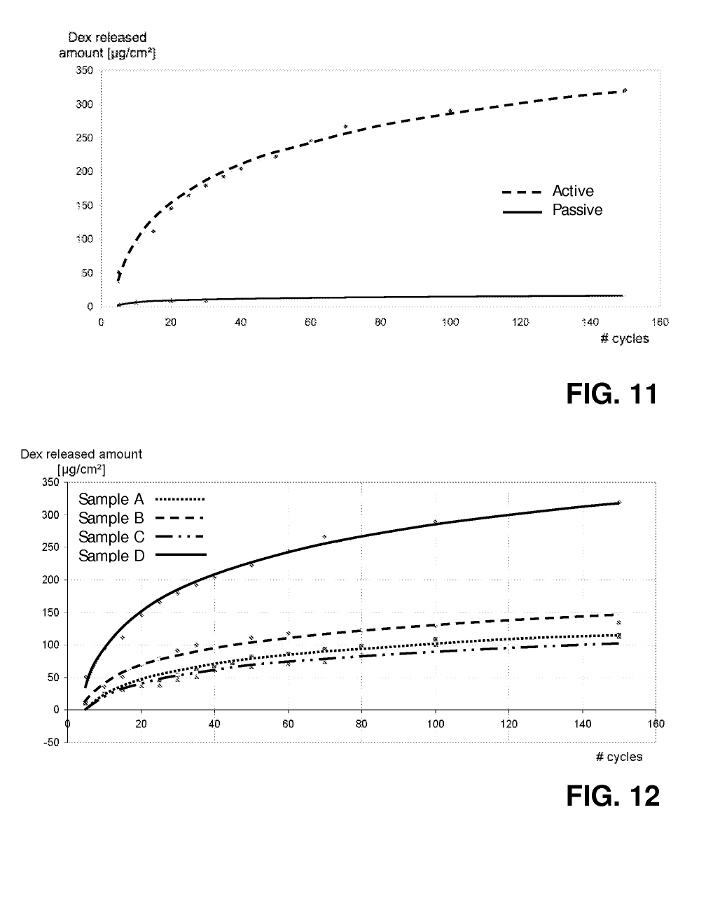

The amount of dexamethasone released via electrical stimulation of the array was measured by UV-Vis absorption and compared with the amount of dexamethasone released in the absence of electrical stimulation of the array. When electrical stimulation was employed, it was carried out by cyclic voltammetry with a terminal potential of −0.8 V to +0.9V and a scanning speed of 100 mV/s. When no electrical stimulation was employed, the amount of dexamethasone released was measured after 5, 10, 20 and 30 minutes after contact with a solution of PBS. Considering a minute is a potential cycle at 100 mV/s, these time periods were set parallel with the periods of release during electrical stimulation. FIG. 11 shows a curve of the amount of dexamethasone released via electrical stimulation (active release), the latter being compared to passive release (i.e. without electrical stimulation) over time/cycles. These results indicate that the amount of dexamethasone released by electrical stimulation is well above the amount released passively and that active release follows a kinetic order of one.

4. Comparison of Nanowire Array Electrodes with Non-Nanowire Array Electrodes

A comparison with samples without nanostructures was carried out to determine the interest of the nanowires for controlled release and the holding of the coating during use of the electrode. This study indicates that the presence of nanowires strongly influences the electroactivity of the coating. The depositing of polypyrrole on a nanostructured metal surface increased electroactivity coating; this phenomenon is linked to an increase in electrical conductivity of the polypyrrole. It is also important to note that the nanostructuring improves adherence of the film and increases the specific surface of the electrode.

### 5. Thickness of the Coating

The film thickness of polypyrrole affects the amount of therapeutic composition incorporated that can be released. The result shown in FIG. 12 demonstrates that thicker films (Sample D; load consumed during the electropolymerisation=70 mC/cm²) allowed the release of a greater amount of dexamethasone compared with thinner films (Samples A to C). Moreover, it is important to note that the tests are reproducible (Samples A to C: PPy films/DEX synthesized in similar experimental conditions: load consumed during the electropolymerisation=30 mC/cm²). In the case of thinner films, the amount of dexamethasone released after 150 cycles was 121±12 micrograms/cm². It reached 300 micrograms/cm² for the thicker films.

### 6. Conclusions

This study aimed to develop a process for making nanostructured electrodes modified for release of anti-inflammatory molecules and studying the potential contributions of nanoscale structures with the characteristics of the electrodes. The various stages of the manufacturing process were developed and demonstrate the reproducibility of manufacturing nanostructured electrodes. The manufacture of these electrodes was in gentle conditions that respects the needs of industries and biomedical and pharmaceutical applications.

The study of the performance of electrodes highlighted the influence of the nanostructure on the electrical behavior of the electrodes (increase of electroactivity, increasing specific surface area and improving the adhesion of PPy film onto the metal support). In addition, a kinetic order of one for the release of biomolecules has been revealed, and the influence of thickness on the performance of electrodes was demonstrated. The combination of nanostructuring phenomenon with the release of biomolecules with a film of polypyrrole therefore has a synergistic effect on the release.

### 7. Fabrication of a Porous Template on Top of Platinum Foil

The polymer solution is prepared from PC pellets (Lexan 145 from General Electric) dissolved in chloroform at a concentration from 3 to 9% and spin-coating is therefore performed at a velocity from 1000 to 6000 rpm depending on the required final thickness (from 200 nm to several μm).

Afterwards, the supported PC layer is irradiated with energetic heavy ions through a mask to limit the creation of linear damaged tracks above the Pt contacts only. Heavy ion irradiation is performed under vacuum or in air with e.g. Ar, Kr or Xe (typical energy in the range 1 to 6 MeV/amu) at a defined fluence between 1.105 and 4.109 cm-2.

Prior to the etching, irradiated PC layer is UV sensitised with a UVA or a UVB source for 1 to 4 hours to increase the track etching selectivity. This treatment remains a critical step in the process as it significantly influences the final pore size and shape of the track etched templates.

Etching is performed in a temperature-regulated bath filled with a 0.5 N or a 2.0 N NaOH aqueous solution at a temperature up to 70° C. and for a time up to 4 hours depending on the final requested pore size. Methanol (from 10 to 50% v/v) can be added in the etching solution as it improves the final adhesion of the PC layer after etching; in this case, etching time is appropriately adjusted and etching bath temperature is limited to 50° C. A surfactant is also added in the etching solution to ensure a homogeneous etching. After etching, the samples are cleaned in successive baths containing an acetic acid aqueous solution (15 wt %), a 10 to 50 v/v methanol aqueous solution and demineralised water at a temperature adjusted between room temperature and 70° C. Samples are then dried using filtered nitrogen flux. By this way, true nanopores as small as 10 nm can be obtained.

Samples are therefore characterised; pore size is controlled using a scanning electron microscopy (SEM-LEO 982) which allows the surface observation of the template at very low voltage under conditions where no metallic coating is required; pore size as small as 15 nm can be observed.

### 8. Fabrication of Therapeutic Composition-Modified Polypyrrole Nanostructured Electrodes

The objective is to use the polymeric nanoporous layer deposited on top of the platinum bounded contacts as template to prepare electroactive conjugated polymer nanostructures.

Two strategies to prepare polypyrrole (PPy) nanostructured electrodes for controlled and local release of anti-inflammatory therapeutic composition can be used:

A) First configuration device (FIG. 1) where the therapeutic composition is directly incorporated into a thin polypyrrole layer electropolymerized at the surface of a metallic nanowire array.

Pyrrole (99%, Acros) was purified immediately before use by passing it through a micro-column constructed from a Pasteur pipette, glass wool and activated alumina. De-ionised water was used to prepare all aqueous solutions. All electrochemical experiments are performed with a potentiostat/galvanostat EG&G Princeton Research 273A in a one-compartment.

Platinum plating solution is made in-house from 0.01 M Na₂PtCl₆.6H₂O, 0.5 M H₂SO₄ in de-ionised water. Pt is electrodeposited potentiostatically at −0.2 V within the pores of the polycarbonate nanoporous layer deposited on top of the platinum bounded contacts. The polycarbonate nanoporous template is removed by dissolution into dichloromethane. Electropolymerisation of pyrrole is then carried out in water in presence of the therapeutic composition (for instance, dexamethasone disodium phosphate or anti-TNF-alpha) on the Pt nanowire array present at the electrode surface. Electrosynthesis of polypyrrole is carried out by chronoamperometry at a constant applied potential of 0.8 V or by cyclic voltammetry (CV) by repeated scans over the 0 to 0.8 V potential range at different scan rates.

A) Second Configuration Device (FIG. 2) where the Therapeutic Composition is Immobilised within Polypyrrole Micro- or Nano-Containers:

Pyrrole (99%, Acros) is purified immediately before use by passing it through a micro-column constructed from a Pasteur pipet, glass wool and activated alumina. Lithium perchlorate (LiClO4, Janssen Chemical) is used without any prior purification. De-ionised water was used to prepare all aqueous solutions. All electrochemical experiments were performed with a potentiostat/galvanostat EG&G Princeton Research 273A in a one-compartment cell at room temperature with a platinum disc counter electrode and Ag/AgCl reference electrode. Electropolymerisation of pyrrole is carried out in water in presence of 0.1 M LiClO₄ within the pores of the template deposited on top of the platinum bounded contacts. Electrosynthesis of polypyrrole is carried out either by chronoamperometry at a constant applied potential of 0.8 V or by cyclic voltammetry (CV) by repeated scans over the 0 to 0.8 V potential range at different scan rates. The resulting polypyrrole micro- or nano-containers are then filled with the therapeutic composition. (for instance, dexamethasone disodium phosphate (Sigma) or anti-TNF-alpha). The filled micro- or nano-containers are then closed by electrodeposition of a thin polypyrrole layer on top of them.

At each step of their fabrication, the morphology of the samples are characterised by scanning electron microscopy (SEM-LEO 982)

### 9. Fabrication of the Drug Delivering Self-Sizing Spiral Cuff Electrodes

A: The basic fabrication of this electrode takes place as described in Naples et al. “A spiral nerve cuff electrode for peripheral nerve stimulation”. IEEE Trans Biomed Eng, 1988; 35(11): 905-916 and U.S. Pat. No. 4,602,624. The main changes from the known process can be described as follows. Prior to insertion in one of the two silicone sheets (Nusil med 4750) that will form the electrode, some or all of the platinum contacts (99.95% purity platinum foil, 25 μm thick, Alfa Aesar, Germany) already bonded to a steel wire (316LVM multistrand stainless steel insulated with fluorinated ethylene-propylene polymer from Fort Wayne Metals, Fort Wayne, USA) are processed as indicated in example 1 and 2. Thereafter, the contacts are mounted on one of the silicone sheets. The contacts are coated with a protective layer and the usual gluing process is performed with the second silicone sheet. This second sheet is stretched or not according to the type of electrode to be made, either a spiral cuff nerve electrode or a flat sheet multicontact electrode. Finally, windows must be cut out through the silicone layer covering in front of the contact and the protective coating eliminated. Cutting out the window is facilitated by the fact that the density of nanostructure is preferably much higher in the middle of the contact area than around the margin of it. Cutting the windows by laser is a satisfactory alternative.

B: An alternative to the fabrication method above involves the use of metallized (platinum) tracks on one of the silicone sheets (Vince et al “Biocompatibility of platinum-metallized silicone rubber: in vivo and in vitro evaluation”. J Biomater. Sci Polym. Ed, 2004; 15(2): 173-188.). The procedures of examples 1 and 2 must now be slightly modified to be applied to a larger metallized silicone sheet instead of isolated metal contacts. Everything else remains identical except that the window cutting should now preferably be done with the laser procedure.

1. A nanowire array for electrically-controlled elution of a therapeutic composition comprising a plurality of nanoscopic-sized wires, nanowires, attached to an electrically conducting solid support, said nanowires formed from electroactive conjugated polymer containing or doped with said therapeutic composition coated over a plurality of nanoscopic sized electrically conducting metallic protrusions.

2. Array according to claim 1, configured such that a plurality of nanowire wires densities or sizes is used in different regions of the contact area in order to compensate for the edge effect so that the current becomes uniform over the area and the overall current a contact safely delivers becomes much higher.

3. Array according to claim 1, configured such that the ratio of the total area to the area of the electrically conducting solid support is greatest at the centre of the array, allowing compensation for non-uniform current density at the array surface.

4. Array according to claim 1, wherein a nanowire of said array has an elongate shape having a width between 10 nm and 10 microns.

5. Array according to claim 1, wherein a nanowire, of said array has an aspect ratio (length/width) between 0.4 and 2000.

6. Array according to claim 1, wherein a nanowire is oriented essentially perpendicular to a surface of the electrically conducting solid support.

7. Array according to claim 1, wherein said electroactive conjugated polymer is formed from monomers of any of pyrrole or substituted pyrrole derivatives, aniline or substituted aniline furan or substituted furan derivatives, thiophene or substituted thiophene derivatives, phosphole or substituted phosphole derivatives, silole or substituted silole derivatives, arsole or substituted arsole derivatives, borole or substituted borole derivatives, selenole, substituted selenole derivatives or aniline and substituted aniline derivatives.

8. Array according to claim 1, wherein the electroactive conjugated polymer is a polymer comprising a compound of formula (I) or (II) 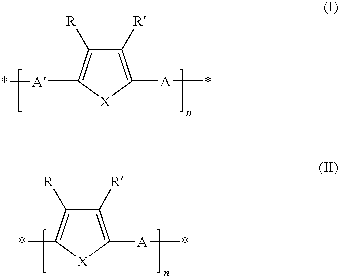
wherein
n is an integer greater than or equal to 3,
X is selected from the group consisting of —NR¹—, O, S, PR², SiR⁵R⁶, Se, AsR³, BR⁴ wherein R′, R², R³, R⁴, R⁵ and R⁶ are independently selected from the group consisting of hydrogen, alkyl or aryl group,
R and R′ are independently selected from the group consisting of alkyl, aryl, hydroxyl, alkoxy or R and R′ together with the carbon atoms to which they are attached form a ring selected from aryl, heteroaryl, cycloalkyl, heterocyclyl, and
A and A′ are independently selected from the group consisting of heterocyclyl, alkenyl, alkynyl or aromatic ring.

9. Array according to claim 1, wherein said electroactive conjugated polymer is a polypyrrole.

10. Array according to claim 1, wherein said nanoscopic sized electrically conducting protrusions are formed from copper, titanium, gold, silver, platinum, palladium, bismuth, or nickel.

11. Array according to claim 1, where said nanoscopic sized electrically conducting protrusions are of suitable size and shape to provide, after coating with electroactive conjugated polymer doped with said therapeutic composition, a nanowire having an elongate shape having a width between 10 nm and 10 microns and an aspect ratio (length/width) between 0.4 and 2000.

12. Array according to claim 1, wherein said electrically conducting solid support is made from any of copper, titanium, gold, silver, platinum, palladium, bismuth, nickel, stainless steel, preferably platinum.

13. Array according to claim 1, wherein said therapeutic composition comprises one or more nutritional substances including vitamins, antioxidants or minerals.

14. Array according to claim 1, wherein said therapeutic composition comprises at least one TNF-alpha inhibitor.

15. Array according to claim 14, wherein said TNF-alpha inhibitor is any of adalimumab, infliximab, etanercept, certolizumab pegol, or golimumab.

16. Array according to claim 1, wherein said therapeutic composition comprises at least one anti-inflammatory agent.

17. Array according to claim 16, wherein said anti-inflammatory agent is any of dexamethasone disodium, aceclofenac, acemetacin, aspirin, celecoxib, dexibuprofen, dexketoprofen, diclofenac, diflunisal, etodolac, etoricoxib, fenbrufen, fenoprofen, flurbiprofen, ibuprofen, indomethacin, ketoprofen, ketorolac trometamol, lumiracoxib, mefanamic acid, meloxicam, nabumetone, naproxen, nimesulide, oxaprozin, parecoxib, phenylbutazone, piroxicam, proglumetacin, sulindac, tenoxicam or tiaprofenic acid.

18. Array according to claim 1, wherein said therapeutic composition comprises an active compound that is an anticancer drug, antipsychotic, antiparkinsonians agent, antiepileptic agent, or antimigraine agent.

19. Array according to claim 1, wherein said therapeutic composition comprises nucleic acids such as nucleotides, oligonucleotides, antisense oligonucleotides, DNA, RNA and mRNA; amino acids and natural, synthetic and recombinant proteins, glycoproteins, polypeptides, peptides, enzymes, antibodies, hormones, cytokines and growth factors.

20. (canceled)

21. An electrode contact wherein the electrically conducting solid support of the array as defined in claim 1 is formed from at least part of said electrical contact.

22. An electrical-stimulation or -recording electrode incorporating an electrode contact as defined in claim 21.

23. Electrode according to claim 22, configured as a cuff electrode.

24. Electrode according to claim 22, configured as a vagus nerve stimulation and/or recording electrode.

25. Electrode according to claim 22, configured as a peripheral nerve stimulation and/or recording electrode.

26. Electrode according to claim 22, configured as a deep brain stimulation and/or electrode.

27. Electrode according to claim 22, wherein said electrode is incorporated into a cochlear implant.

28. Electrode according to claim 22, configured as a brain stimulation and recording electrode.

29. Electrode according to claim 22, configured as a tumour implantable device.

30. Electrode according to claim 22, configured as a subcutaneously implantable device.

31. Electrode according to any of claim 22, incorporated into a visual prosthesis.

32. Method for the preparation of a nanowire array for eluting a therapeutic composition comprising the steps of:
(a) depositing a layer of polymeric matrix onto at least part of an electrically conducting solid support,
(b) creating pores in the layer of polymeric matrix by track-etching so forming a polymeric nanoporous layer,
(c) electrodepositing an electrically conducting metallic material within the pores the polymeric nanoporous layer,
(d) dissolving the polymeric nanoporous layer to form electrically conducting metallic protrusions, and
(e) electropolymerising onto said protrusions an electroactive conjugated polymer doped with therapeutic composition;
so forming a nanowire array.

33. Method according to claim 32, wherein a plurality of nanowire wires densities or sizes is used in different regions of the contact area in order to compensate for the edge effect so that the current becomes uniform over the area and the overall current a contact safely delivers becomes much higher.

34. Method according to claim 32, wherein the ratio of the total area to the area of the electrically conducting solid support is greatest at the centre of the array, allowing compensation for non-uniform current density at the array surface.

35. Method according to claim 32, wherein
the electrically conducing solid support is configured such that the ratio of the total area to the area of the electrically conducting solid support is greatest at the centre of the array, allowing compensation for non-uniform current density at the array surface,
the electroactive conjugated polymer is formed from monomers of any of pyrrole or substituted pyrrole derivatives, aniline or substituted aniline furan or substituted furan derivatives, thiophene or substituted thiophene derivatives, phosphole or substituted phosphole derivatives, silole or substituted silole derivatives, arsole or substituted arsole derivatives, borole or substituted borole derivatives, selenole, substituted selenole derivatives or aniline or substituted aniline derivatives,
the therapeutic composition comprises one or more nutritional substances including vitamins, antioxidants or minerals, TNF-alpha inhibitor, anti-inflammatory agent, an active compound that is an anticancer drug, antipsychotic, antiparkinsonians agent, antiepileptic agent, or antimigraine agent, nucleic acids such as nucleotides, oligonucleotides, antisense oligonucleotides, DNA, RNA and mRNA, amino acids and natural, synthetic and recombinant proteins, glycoproteins, polypeptides, peptides, enzymes, antibodies, hormones, cytokines and growth factors, and
the conducting protrusion, wherein said conducting protrusions are formed from copper, titanium, gold, silver, platinum, palladium, bismuth, or nickel.

36. Method for preparing an electrode contact comprising the method of claim 32, where-in the electrically conducting solid support is formed by at least part of a contact of the electrode.

37. A method for preparing a spiral cuff electrode having an inward facing surface disposed with an electrode contact, and an outward facing surface, said method further comprising the steps of:
bonding one surface of an unstretched flexible sheet to one surface of a second flexible sheet wherein the second flexible sheet has been stretched or not in one direction prior to bonding, and
providing an electrode contact using the method as defined in claim 32 located on or in the inward facing surface, so forming a spiral cuff electrode or a flat sheet electrode.

38. Method according to claim 37, wherein the flexible sheets are made from a silicone elastomer, preferably silicone rubber.

39. Method according to claim 37, wherein the contact electrode is provided between the bonded sheets, and is exposed by an opening in the stretched flexible sheet.
